(19) United States
(12) Patent Application Publication
(10) Pub. No.: US 2022/0288213 A1
(43) Pub. Date: Sep. 15, 2022

(54) INSULIN ANALOGUES WITH GLUCOSE REGULATED CONFORMATIONAL SWITCH

(71) Applicant: Thermalin Inc., STONEHAM, MA (US)

(72) Inventor: Jeremy W. GLEATON, Cleveland, OH (US)

(21) Appl. No.: 17/630,258

(22) PCT Filed: Jul. 31, 2020
(86) PCT No.: PCT/US20/44415

§ 371 Date: Jan. 26, 2022

Related U.S. Application Data
(60) Provisional application No. 62/934,774, filed on Nov. 13, 2019.
(60) Provisional application No. 62/931,933, filed on Nov. 7, 2019.
(60) Provisional application No. 62/880,945, filed on Jul. 31, 2019.

(51) Int. Cl. A61K 47/54; C07K 14/62; A61K 47/64

(52) U.S. Cl. CPC A61K 47/545; C07K 14/62; A61K 47/64

(57) ABSTRACT

The present invention relates to glucose-responsive insulin analogues, compositions including the glucose-responsive insulin analogues, and methods of lowering blood sugar of a patient using the insulin analogue or compositions thereof.

### TECHNICAL FIELD

The present invention relates to glucose-responsive insulin analogues. The invention is also related to compositions comprising the glucose-responsive insulin analogue and a method of controlling blood sugar levels in a patient by administering the glucose-responsive insulin analogues to the patient.

### BACKGROUND

Hyperglycemia is a condition in which an excessive amount of glucose circulates in an individual's blood plasma. This condition generally results when a patient has a blood glucose level of 10 mmol/L (180 mg/dl) or greater, but symptoms and effects may not start to become noticeable until greater blood glucose concentrations are reached, such as 15 to 20 mmol/L (270 to 360 mg/dl) or greater. The long-term adverse effects of hyperglycemia include blindness, loss of kidney function, nerve damage, loss of sensation, and poor circulation in the periphery potentially requiring amputation of the extremities.

As patients with Type 1 diabetes do not produce insulin, the primary treatment for Type 1 diabetes is multiple daily insulin injection therapy. The treatment of Type 2 diabetes typically starts with management of diet and exercise but it is often not an effective long-term solution. When diet and exercise are no longer effective, various non-insulin oral medications are prescribed that act by increasing the amount of insulin produced by the pancreas. These treatments are limited in their ability to manage the disease effectively and generally have significant side effects, such as weight gain and hypertension.

Because of the limitations of non-insulin treatments, many patients with Type 2 diabetes eventually require insulin therapy. However, currently known insulin therapies often lead to recurrent hyperglycemia in the patient because of unpredictable variability in the patient's blood glucose levels and the lack of adequate feedback mechanisms that match the level of insulin with the level of blood glucose. Another problem with the existing insulin treatments is that they are insensitive to the variance in, e.g., a patient's diet, exercise regimen, stress, and several other factors that can result in fluctuations of blood glucose.

Currently known long acting insulins typically start to lower blood glucose about one hour after injection. While long acting insulins cover an average patient's basal insulin needs, they often do not last long enough. This causes the patient to become hyperglycemic when the concentration of long acting insulins recedes in the body. Additionally, long acting insulins do not adjust the amount of insulin released from the formulation based on the patient's real time needs. In some instances, it may release less insulin than needed by a patient to reduce the blood glucose levels, thus, causing hyperglycemia.

With rapid-acting insulins, a patient's insulin levels depend on the time and quantity of insulin injected with peak insulin levels typically occurring within 50 to 70 minutes following the injection. Peak plasma levels of the rapid acting insulin are independent of the blood glucose levels and they do not respond to increased blood glucose levels in a patient. Thus, if a patient underestimates his/her blood glucose levels, rapid acting insulin formulations are not able regulate the patient's blood glucose levels and, as consequence, the patient may become hyperglycemic. Also, a patient cannot always administer insulin dosages properly and even when insulin injections are properly administered, they do not replicate the natural glucose feedback profile of insulin. Often, injected insulin enters the blood slowly, with no regard to the current blood glucose level.

Thus, there is a need in the art to find ways to mimic the natural feedback mechanism that allows blood insulin levels to rise or fall based on the glucose level. It is an objective of the present invention to provide a glucose-responsive insulin for regulating blood glucose levels in patients with Type 1 or Type 2 diabetes.

### SUMMARY OF THE INVENTION

The present invention is related to novel insulin analogues that bind to glucose and provide glucose-responsive binding to the insulin receptor thus regulating the level of glucose in blood. The insulin analogues of the present invention have at least two states: 1) a closed (or switched off) state where the insulin analogue has a reduced binding affinity for its receptor and 2) an open (or switched on) state where glucose is bound to the insulin analogue and the binding affinity of the insulin analogue to its receptor is restored or increased as compared to the switched off state.

This restoration or increase of binding affinity of the insulin analogues of the present invention is responsive to glucose concentration within a subject's blood. When the concentration of glucose in the subject's blood is higher than physiological levels (i.e., hyperglycemic), the insulin analogue binds to glucose and becomes switched on. In this switched on state, the insulin analogue binds to its receptor and regulates concentration of blood glucose. When the concentration of glucose in the subject's blood is normal or lower than normal, then the insulin analogue remains in switched off state. In this switched off state, the insulin analogue has lower affinity for its receptor as compared the switched on state.

These insulin analogues of the present invention need at least two elements in order to properly function in a glucose responsive manner. The first element is a glucose binding moiety and the second element is a binding partner moiety that binds to the glucose binding moiety. One or both these moieties are linked to the insulin analogue. The glucose binding moiety and/or the binding partner moiety are linked to the insulin analogue in a manner such that they can interact with one another, optionally in a reversible manner. Interaction between the two elements reduces the binding affinity of the insulin analogue to its receptor.

Thus, when the concentration of glucose is higher than normal, the interaction between the glucose-binding moiety and the binding partner moiety is disrupted due to increased competition by glucose for binding to the glucose binding moiety. This disruption leads to an increase or restoration of the insulin analogue's binding affinity for its receptor. Thus, the intramolecular interaction between the glucose-binding moiety and the partner moiety switches “off” the insulin analogue whereas disruption of the interaction switches the insulin analogue “on.”

In various embodiments, the glucose binding moiety of the present invention is a multivalent aromatic boronic acid, such as a divalent aromatic boronic acid. These aromatic boronic acids can exhibit selective binding to D-glucose. This enhanced sensitivity of multivalent boronic acids for glucose is surprisingly different from monomeric boronic acids. Monoboronic acids promiscuously bind to other saccharides, thus, making it difficult to differentiate between saccharides. For example, monoboronic acids exhibit inherent fructose selectivity among monosaccharides (J. P. Lorand and J. O. Edwards, J. Org. Chem., 1959, 24, 769-774, which is hereby incorporated by reference in its entirety). The divalent and multivalent aromatic boronic acids of the present disclosure show selectivity for glucose because they provide proper orientation and presentation of the boronic acid groups to the hydroxyl groups of a glucose molecule.

In another aspect, the present invention provides a composition comprising the insulin analogue as disclosed herein and a pharmaceutically acceptable carrier or excipient. In yet another aspect, the present invention provides a method of lowering the blood sugar of a patient comprising administering a physiologically effective amount of the insulin analogue as disclosed herein or a composition thereof.

Other aspects and embodiments of the invention will be apparent from the following detailed description.

### BRIEF DESCRIPTION OF THE DRAWINGS

FIG. 1A is a schematic representation of the sequence of human proinsulin including the A- and B-chains and the connecting region shown with flanking dibasic cleavage sites (filled circles) and C-peptide.

FIG. 1B is a structural model of proinsulin, consisting of an insulin-like moiety and a disordered connecting peptide (dashed line).

FIG. 1C is a schematic representation of the sequence of human insulin including the A-chain and the B-chain and indicating the position of residues A6, A7, A11, A20, B7, B19, B27, and B30 in the B-chain.

FIG. 2A is a schematic representation of one embodiment of a glucose-responsive insulin analogue. It shows the glucose binding moiety and the binding partner modification, e.g., a polyol. The A-chain is represented by the shorter horizontal cylinder and the B-chain by the longer horizontal cylinder. The canonical disulfide bridges of wild-type insulin are indicated by black lines (see box at bottom right). The A-chain is modified by a glucose-binding moiety and spacer at or near its N-terminus (V shape and wavy line, respectively). The B-chain is modified by one or more polyol moieties and spacer at or near its C-terminus (circle and spacer element, respectively).

FIG. 2B is a schematic representation of another embodiment of a glucose-responsive insulin. It also shows the glucose binding moiety and the polyol modification as the binding partner. The A-chain is represented by the shorter horizontal cylinder and the B-chain by the longer horizontal cylinder. The canonical disulfide bridges of wild-type insulin are indicated by black lines (see box at bottom right). The A-chain is modified by one or more polyol moieties and spacer at or near its N-terminus (circle and spacer element, respectively). The B-chain is modified by a glucose-binding moiety and spacer at or near its N-terminus (V shape and wavy line, respectively).

FIGS. 2C, 2D, 2E and 2F illustrate embodiments of the glucose-responsive insulin analogue in which the binding partners (e.g., the glucose-binding moiety and a binding-partner element) are both conjugated at or near the end of the B-chain, optionally with spacer elements to allow glucose-responsive binding to occur between the binding partners. Reversible interaction between the binding moieties inhibits affinity for the insulin receptor. Disruption of the interaction between the binding moieties by glucose binding to the glucose-binding moiety restores affinity for the insulin receptor. Those skilled in the art will recognize that this approach is not limited to the C-terminus of the B-chain.

FIGS. 2G and 2H illustrates an embodiment of the glucose-responsive insulin analogue in which the insulin analogue is conjugated at or near each termini with a binding moiety (a binding partner element is illustrated in FIG. 2G and a glucose binding moiety is illustrated in FIG. 2H). Glucose-responsive binding is formed at both ends by the respective binding partner (e.g., glucose-binding moiety) linked by a sufficiently large spacer.

FIG. 2I illustrates embodiments of the invention employing inhibitory moieties, which may include, for example, macromolecular carriers such as albumin or albumin-binding moiety, a lectin-binding moiety, or a peptidic or other moiety that masks receptor binding surfaces. The binding of these inhibitory moieties can be disrupted in the presence of glucose and the inhibitory moieties can be tethered to the insulin analogue (right panel) or non-tethered (left panel).

FIG. 3 shows the results obtained from the Alizarin Red S Assay.

FIG. 4 illustrates a scheme for the solid phase synthesis of a glucose-binding moiety attached to a peptide (DA-Z4 KB28 PB29).

FIGS. 5A and 5B show the results obtained for example glucose-responsive insulins in the insulin receptor activation assay with and without glucose.

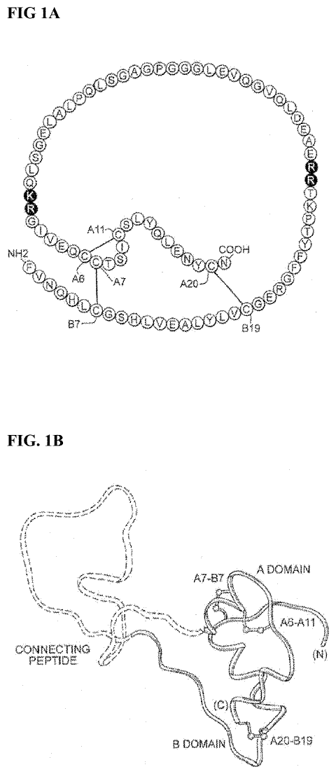

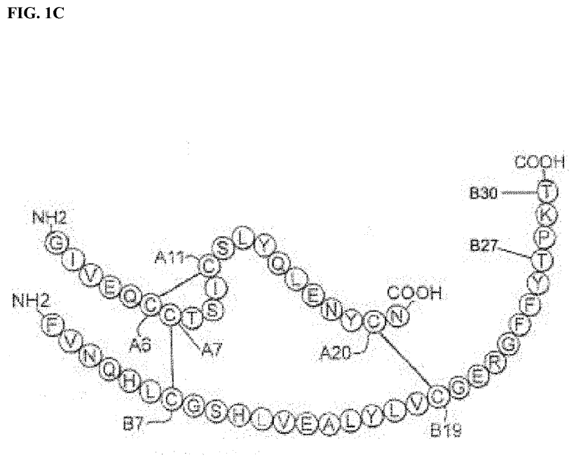

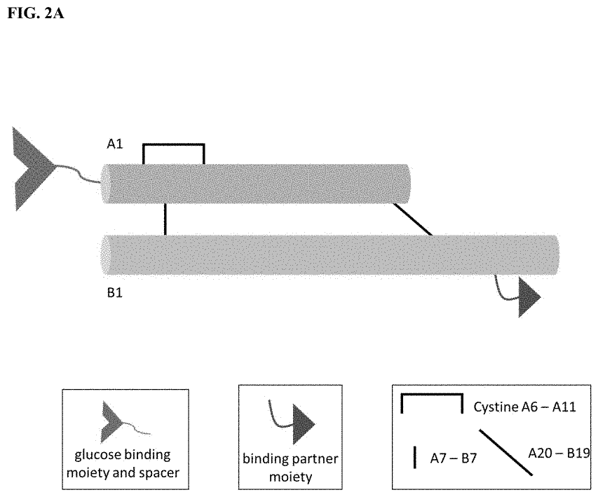

### DETAILED DESCRIPTION

The present invention is related to modified insulin analogues that have an enhanced ability to provide glycemic control through glucose-dependent binding to insulin receptors. The modified insulin analogues, disclosed herein, have low affinity for insulin receptors, when the concentration of blood glucose is within or lower than the normal physiological range. This reduction in the ability of the modified insulin analogue to bind to its receptor is due to an interaction between a glucose-binding moiety and a binding partner moiety, at least one of which is derivatized to the insulin analogue. This interaction between the partner moiety and the glucose binding moiety changes the conformation of the insulin analogue (switched “off” state) such that affinity for the insulin receptor is reduced. This interaction between the glucose-binding moiety and the binding partner moiety is disrupted in the presence of glucose in a higher than normal physiological range.

The binding affinity of the modified insulin molecule is restored or increased, as compared to switched “off” state, when the glucose level in a subject's blood is higher than normal (or the subject is hyperglycemic). Under hyperglycemic conditions, glucose competes for binding to the glucose binding moiety present on the insulin analogue and displaces the intramolecular partner moiety that interacts with the glucose binding moiety in the switched “off” state of the insulin analogue. This displacement of the partner moiety restores the binding affinity of the insulin analogue to its receptor. Therefore, the insulin analogue of the present invention exhibits a glucose responsive binding to its receptor. The restoration of insulin analogue's binding to its receptor provides a means to control the level of glucose in the subject's blood.

Therefore, in various embodiments, the invention provides a glucose-responsive insulin analogue or complex. The analogue or complex comprises an insulin analogue, one or more multivalent boronic acid glucose-binding moieties, and one or more binding partner moieties binding the multivalent boronic acid moiety in the absence of glucose, wherein the glucose-responsive insulin analogue or complex has low affinity (compared to wild type insulin) for the insulin receptor in the absence of glucose.

In some embodiments, the multivalent boronic acid moiety comprises one or more aromatic boronic acids, and which may include one or more substituent-modified aromatic boronic acids. In one embodiment, the substituent is a halogen. In another embodiment, the substituent can be a cyano, nitro, amino, methoxy, ethoxy, carboxamide, and/or a trifluoromethyl group. In some embodiments, the multivalent boronic acid moiety is a divalent boronic acid moiety (e.g., a divalent aromatic boronic acid moiety, which may be halogen modified in its aromatic ring). In some embodiments, the multivalent boronic acid moiety is a benzoxaborole derivative.

According to the present invention, an aromatic boronic acid moiety having one boronic acid is defined as monovalent. An aromatic boronic acid is divalent when it has two boronic acid moieties. This divalent aromatic boronic acid can form 4 or 6 boronate esters. An aromatic boronic acid is multivalent when it has two or more boronic acid moieties.

The multivalent and divalent boronic acid moieties may further comprise a linker, which is optionally linked by an amide or peptidic linker, an acyl linker (e.g., a functionalized hydrocarbon linker), an aromatic linker, a heterocyclic linker, or a PEG linker. In some embodiments these linkers are spaced such that they allow for the size of a D-glucose molecule (4-20 Å). For example, in some embodiments, two or more aromatic boronic acids moieties are linked together by one or more linkers to form a divalent aromatic boronic acid or a multivalent boronic acid. In some embodiments, the divalent aromatic boronic acid includes the same aromatic boronic acid moiety, optionally, joined by a spacer element or a linker. In other embodiments, the divalent aromatic boronic acid includes different aromatic boronic acid moieties, optionally, joined by a spacer element or a linker. For example, a halogenated aromatic boronic acid moiety can be linked to non-halogenated aromatic boronic acid moiety or a benzoxaborole moiety can be paired with a phenyl boronic acid in a divalent aromatic boronic acid scaffold. In the context of multivalent aromatic boronic acids of the present invention, the aromatic boronic acid moiety can have the same aromatic boronic acid moiety or a combination of different aromatic boronic acid moieties. In some embodiments, the multivalent aromatic boronic acids have the aromatic boronic acid moieties linked to each other by the same linker or with different linkers. In some embodiments, the multivalent boronic acid linker has 2, 3, 4, or 5 aromatic boronic acids, each optionally linked by a spacer element.

In various embodiments, the insulin analogue or complex comprises a divalent boronic acid moiety of Formula (I):

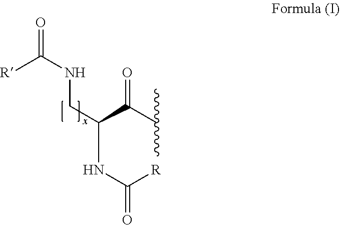

wherein:

X is 1, 2, 3, 4, or 5, and

R and/or R′ comprise a group independently selected from

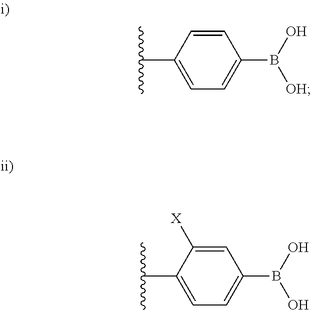

where X is selected from F, Cl, Br, I, —NH₂, —OMe, —NO₂;

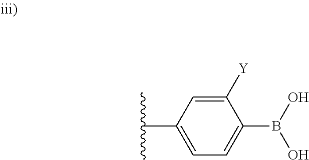

where Y is selected from F, Cl, Br, I, —NH₂, —OMe, —NO₂, —NH(CH₃), —N(CH₃)₂, —(CH₂)ₐNH(CH₃), or —N(CH₂)_{b}(CH₃)₂; a=1, 2, or 3 and b=1, 2, or 3;

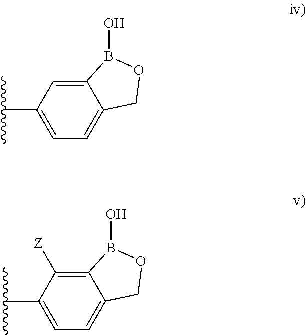

where Z is selected from F, Cl, Br, I, —NH₂, —OMe, —NO₂, and

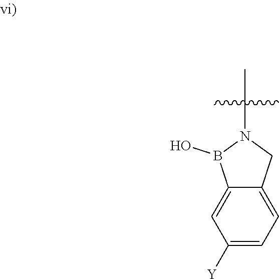

where Y is selected from F, Cl, Br, I, —NH₂, —OMe, —NO₂, —NH(CH₃), —N(CH₃)₂, —(CH₂)ₐNH(CH₃), or —N(CH₂)_{b}(CH₃)₂; a=1, 2, or 3 and b=1, 2, or 3.

For example, the insulin analogue or complex may comprise a divalent boronic acid moiety having the following structure:

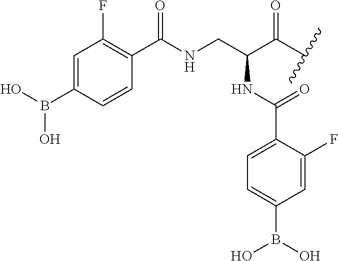 [SMILES: *C(=O)[C@H](CNC(=O)c1ccc(B(O)O)cc1F)NC(=O)c1ccc(B(O)O)cc1F]

In still other embodiments, the insulin analogue or complex comprises a divalent boronic acid moiety having the following structure:

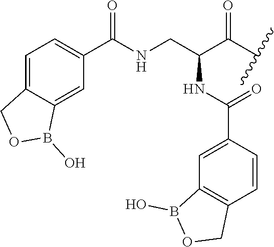 [SMILES: *C(=O)[C@H](CNC(=O)c1ccc2c(c1)B(O)OC2)NC(=O)c1ccc2c(c1)B(O)OC2]

In various embodiments, the insulin analogue or complex comprises a divalent boronic acid moiety of Formula (II):

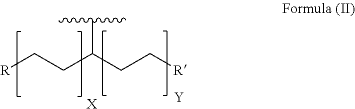

wherein:

X, Y is 1-10, and

R and/or R′ comprises a group independently selected from

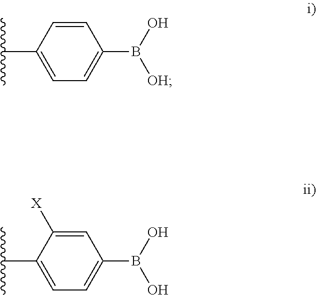

where X is selected from F, Cl, Br, I, —NH₂, —OMe, —NO₂;

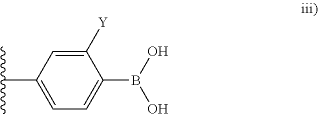

where Y is selected from F, Cl, Br, I, —NH₂, —OMe, —NO₂, —NH(CH₃), —N(CH₃)₂, —(CH₂)ₐNH(CH₃), or —N(CH₂)_{b}(CH₃)₂; a=1, 2, or 3 and b=1, 2, or 3;

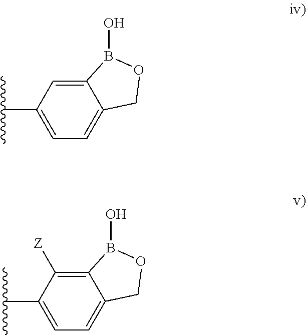

where Z is selected from F, Cl, Br, I, —NH₂, —OMe, —NO₂; and

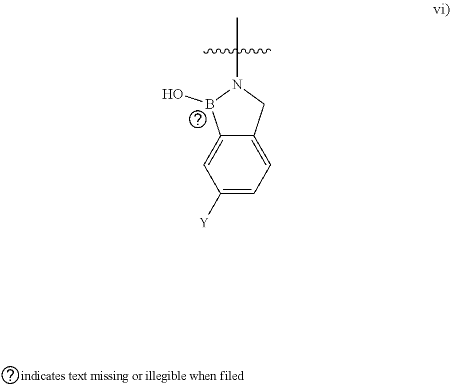

where Y is selected from F, Cl, Br, I, —NH₂, —OMe, —NO₂, —NH(CH₃), —N(CH₃)₂, —(CH₂)ₐNH(CH₃), or —N(CH₂)_{b}(CH₃)₂; a=1, 2, or 3 and b=1, 2, or 3.

In some embodiments, the insulin analogue or complex comprises a divalent boronic acid moiety having a structure of Formula (III):

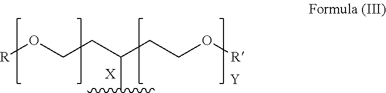

wherein:

X, Y is 1-10, and

R and/or R′ comprises a group independently selected from

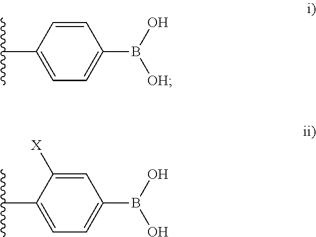

where X is selected from F, Cl, Br, I, —NH₂, —OMe, —NO₂;

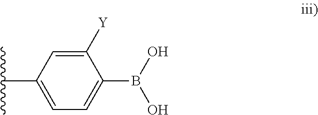

where Y is selected from F, Cl, Br, I, —NH₂, —OMe, —NO₂, —NH(CH₃), —N(CH₃)₂, —(CH₂)ₐNH(CH₃), or —N(CH₂)_{b}(CH₃)₂; a=1, 2, or 3 and b=1, 2, or 3;

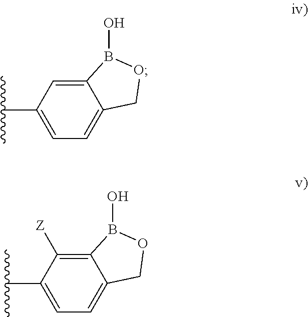

where Z is selected from F, Cl, Br, I, —NH₂, —OMe, —NO₂; and

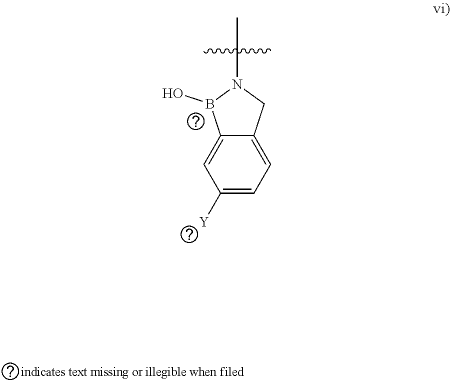

where Y is selected from F, Cl, Br, I, —NH₂, —OMe, —NO₂, —NH(CH₃), —N(CH₃)₂, —(CH₂)ₐNH(CH₃), or —N(CH₂)_{b}(CH₃)₂; a=1, 2, or 3 and b=1, 2, or 3.

In some embodiments, the insulin analogue or complex comprises a divalent boronic acid moiety having a structure of Formula (IV):

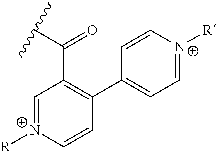

R and/or R′ comprises a group independently selected from

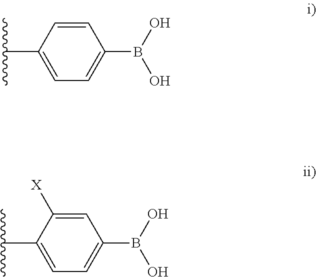

where X is selected from F, Cl, Br, I, —NH₂, —OMe, —NO₂;

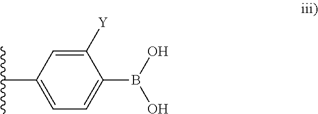

where Y is selected from F, Cl, Br, I, —NH₂, —OMe, —NO₂, —NH(CH₃), —N(CH₃)₂, —(CH₂)ₐNH(CH₃), or —N(CH₂)_{b}(CH₃)₂; a=1, 2, or 3 and b=1, 2, or 3;

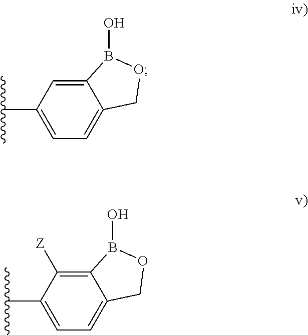

where Z is selected from F, Cl, Br, I, —NH₂, —OMe, —NO₂; and

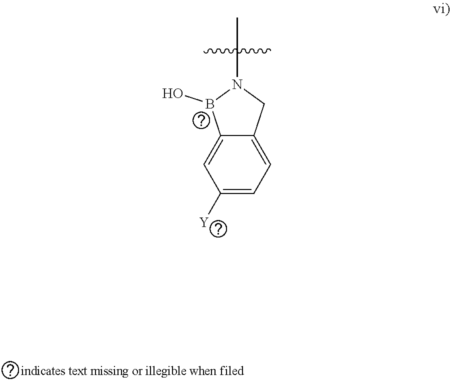

where Y is selected from F, Cl, Br, I, —NH₂, —OMe, —NO₂, —NH(CH₃), —N(CH₃)₂, —(CH₂)ₐNH(CH₃), or —N(CH₂)_{b}(CH₃)₂; a=1, 2, or 3 and b=1, 2, or 3.

Other glucose-binding moieties can be employed, including natural and/or non-natural peptidic glucose-binding moieties, which can be linked to one or more amino acid residues in the insulin analogue, optionally via one or more spacer elements. In some embodiments, the peptidic glucose binding moiety is cyclic. In another embodiment, the peptidic glucose binding moiety is linear. Still other glucose-binding moieties include nucleic acid aptamers, which can be linked to one or more amino acid residues in the insulin analogue, optionally via one or more spacer elements. In various embodiments, the peptidic glucose-binding moiety or nucleic acid aptamer are selective for glucose over other physiologically relevant monosaccharides.

Exemplary glucose-binding moieties are shown in Table 1.

In various embodiments, the binding partner is a polyol-containing moiety. Non-limiting examples of polyol-containing precursor compounds that may be used in the present invention for making the binding partner moieties include 1,3-benzenedimethanol; D-gluconic acid; D-glucoheptanoic acid; Gallic acid (3,4,5-trihydroxybenzoic acid); 3,4-dihydroxybenzoic acid, 3-chloro-4,5-dihydroxybenzoic acid, caffeic acid, rosmarinic acid; 3,4-dihydroxy-5-methoxybenzoic acid; D-glucuronic acid; Shikimic acid; D-(−)-quinic acid; mannitol; fructose; sorbitol; Tris base; 2-(4-aminobutyl)-1,3-propanediol; 3-amino-1-,2-propandiol; 2-aminopropane-1,3-diol; 3-mercaptopropane-1,2-diol; 2-amino-4-pentane-1,3-diol; N-acetyl-D-galactosamine; β-D-galactopyranosylamine; Cafestol; glafenine; glyceraldehyde; glyceric acid; glycerol 3-phosphate; glycerol monostearate; cyclohexane-1,2-diol; cytosine glycol; 4,5-dihydroxy-2,3-pentanedione dihydroxyphenylethylene glycol dithioerythritol; Dithiothreitol; dropropizine; dyphylline; floctafenine; N,N-dimethylsphingosine; tartaric acid; guaifenesin; (3S,4R)-4-methyl-5-hexene-1,3-diol; erythritol; catechol; cyclohexane-1,2-diol; and 1,2-dihydroxybenzene dihydroxyphenylethylene glycol. In some embodiments, the polyol-containing moiety is a natural or synthetically modified monosaccharide, disaccharide, and/or an oligosaccharide. The polyol-containing precursor compounds that may be used to prepare the polyol-containing moiety of the present invention are further described in United States Patent Publication No. 20180057559, which is hereby incorporated by reference in its entirety.

Non-limiting examples of sugar mimetic-containing precursor compounds that may be used in the present invention for making the partner moieties include hexanose, heptanose, and octanose.

In various embodiments, the partner moiety is such that when higher than normal concentration of glucose is present in a subject's blood (e.g., during hyperglycemia), glucose molecules are able to out-compete/displace the partner moiety from its reversible association with the glucose-binding moiety present on the insulin analogue. This dissociation of the partner moiety increases the binding affinity of the insulin analogue to its cognate receptor.

The glucose binding moiety and/or binding partner moiety can be linked to a native amino acid residue or at a mutated (including added, substituted, or a non-standard) amino acid residue of the insulin analogue. In some embodiments, the glucose binding moiety is linked to an alpha-amino functional group or to the side chain amino function of D-homolysine, D-lysine, D-ornithine, D-diaminobutyric acid, D-diaminopropionic acid, L-homolysine, L-lysine, L-ornithine, L-diaminobutyric acid, or L-diaminopropionic acid.

The glucose-binding moiety and/or the binding partner moiety may be amine-linked or thiol-linked to the insulin analogue. For example, where the moiety contains a diol, the diol modification of insulin can be an amine-linked diol modification or an thiol-linked diol modification. Thiol-linked diol modification is formed by thiol functional group of cysteine (or homocysteine) substitutions whereas amine-linked diol modification is provided by the side-chain amino group of lysine, ornithine, diamino-butyric acid or diamino-propionic acid. Where the moiety contains a polyol, the polyol-containing moiety can be linked to insulin at a native amino acid residue or at a mutated (including added, substituted or non-standard) amino acid residue. In some embodiments, the polyol moiety is linked to an alpha-amino functional group or to the side chain amino function of D-lysine, D-ornithine, D-diaminobutyric acid, D-diaminopropionic acid, L-lysine, L-ornithine, L-diaminobutyric acid, or L-diaminopropionic acid.

The invention in various embodiments is applicable to basal as well as prandial insulins. In various embodiments, the insulin comprises A and B chains, or in other embodiments is a single chain insulin. In some embodiments, the insulin analogue is a rapid action or ultra-rapid action insulin analogue, such as those described in U.S. Pat. No. 9,901,622, which is hereby incorporated by reference. In some embodiments, the insulin analogue is a basal insulin, which may optionally have a half-life extension moiety or fusion, such as albumin, elastin-like peptide, acylation or other fusion suitable for extending circulation half-life.

In some embodiments, the insulin analogue or complex comprises an A-chain polypeptide and a B-chain polypeptide, which can be modified with respect to wild-type human insulin.

In some embodiments, the glucose-binding moiety is attached at or near the N-terminus of the A-chain or an extension thereof. A glucose-binding moiety can be attached, e.g., to the first 10 amino acids (i.e., at one or more of positions A1-A10) of the A-chain or an extension thereof. In another embodiment, the glucose-binding moiety can be attached to a single or multiple amino acid extension to the A-chain N-terminus (e.g. A0 position). In other embodiments, the glucose-binding moiety is attached at or near the C-terminus of the B-chain or an extension thereof. For example, the glucose-binding moiety may be attached to any of the last 15 amino acids at the C-terminus (i.e., at positions B16-B30) of the B-chain or an extension thereof. In other embodiments, the glucose binding moiety is attached to an amino acid extension of the B-chain C-terminus (e.g. B31-B34). In some embodiments, multiple glucose binding moieties are attached to any of the above positions on the insulin molecule. In some embodiments, a glucose-binding moiety is attached to one or more positions selected from A0, A1, A4, B16, B25, B26, B27, B28, B29, B30, B31, B32, B33, and B34. In some embodiments, the binding partner moiety is linked to an extension of the B-chain. For example, a glucose-binding moiety can be attached to an extended B-chain at positions B31, B32, B33, B34 or further extended. Extended B-chains insulins may be derived from natural or non-natural amino acids.

In some embodiments, a binding partner moiety is conjugated at one or more amino acids in the A-chain or the B-chain of the insulin analogue. For example, the binding partner moiety can be provided at or near the N-terminus of the A-chain or an extension thereof. In other embodiments, the binding partner moiety can be provided at the C-terminus of the B-chain or an extension thereof. In some embodiments, at least one binding partner moiety is provided within the first 10 amino acid residues from the N-terminus of the A-chain (i.e., one or more of positions A1-A10) or an extension thereof and, in other embodiments, the binding partner moiety is provided within the last 15 amino acids from the C-terminus of the B-chain or extension thereof (i.e., one or more of positions B16-B34). In another embodiment, the binding partner moiety can be attached to a single or multiple amino acid extension to the A-chain N-terminus (e.g. A0 position). In some embodiments, a binding partner moiety is attached to one or more positions selected from A0, A1, A4, B16, B25, B26, B27, B28, B29, B30, B31, B32, B33, and B34. In some embodiments, the binding partner moiety is linked to an extension of the B-chain. For example, a binding partner moiety can be attached to an extended B-chain at positions B31, B32, B33, B34 or further extended. Extended B-chains insulins may be derived from natural or non-natural amino acids.

For example, the A-chain is modified with a glucose-binding moiety and the B-chain is modified with a binding partner moiety; or the B-chain is modified with a glucose-binding moiety and the A-chain is modified with a binding partner moiety. For example, the glucose-binding moiety may be attached at or near N-terminus of the A-chain and the binding partner moiety may be attached at or near C-terminus of the B-chain. Alternatively, the glucose-binding moiety may be attached at or near C-terminus of the B-chain and the binding partner moiety may be attached at or near N-terminus of the A-chain. For example, the glucose-binding moiety may be attached to a position selected from A0, A1-A10 of the insulin analogue, and the binding partner moiety may be attached to a position selected from B16-B30, or in some embodiments, the binding partner moiety is linked to a B-chain extension, such as an extension having additional amino acid positions B31, B32, B33, B34 or further extended. In still other embodiments, the glucose-binding moiety is attached to a position selected from B16-B30 (or an amino acid of a B chain extension), and the binding partner moiety is attached to a position selected from A0, A1-A10. Where there is a B chain extension, in some embodiments, the amino acid at position B31 is cysteine or homocysteine and the amino acid at position B32 is cysteine or homocysteine.

In some embodiments, a glucose-binding moiety or binding partner moiety of the A chain is attached at or near the N-terminus of the A-chain; and the glucose-binding moiety or binding partner moiety of the B chain is attached at or near C-terminus of the B-chain. For example, the glucose-binding moiety or binding partner moiety of the A chain may be attached to a position selected from A0, A1-A10. In some embodiments, the glucose-binding moiety or the binding partner moiety of the B chain is attached to a position selected from B16-B30. In still other embodiments, a glucose binding moiety or binding partner moiety is linked to a B-chain extension, wherein the extension optionally has additional amino acid positions B31, B32, B33, B34 or further extended. For example, the amino acid at position B31 may be cysteine or a homocysteine and the amino acid at position B32 may be cysteine or a homocysteine.

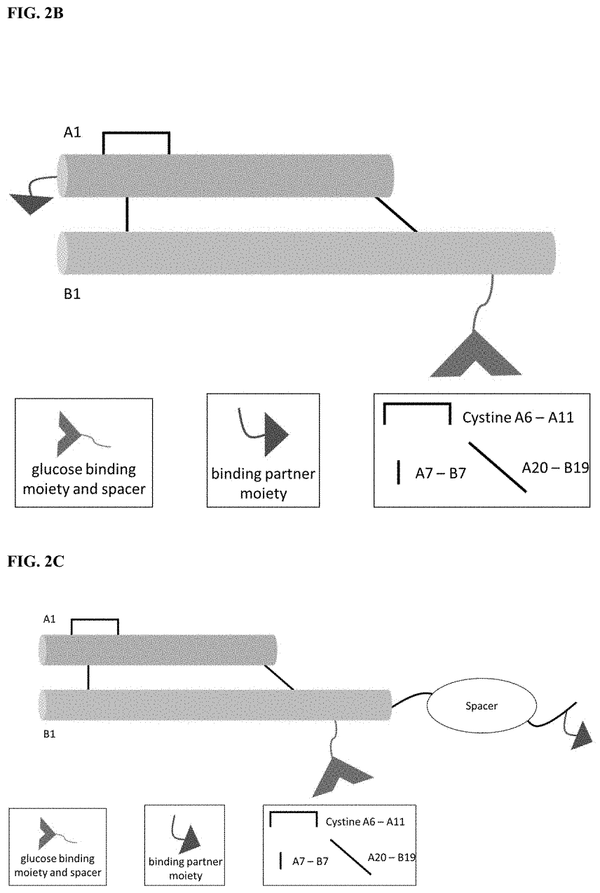

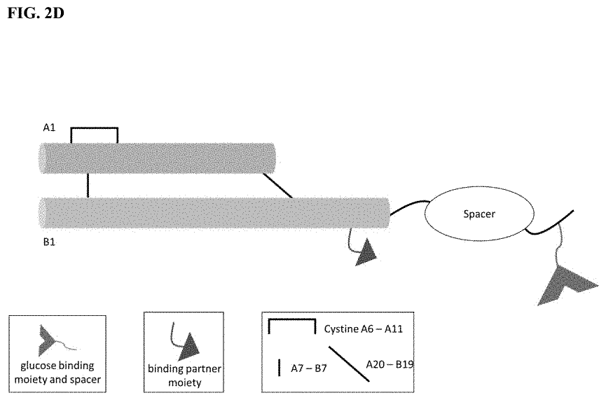

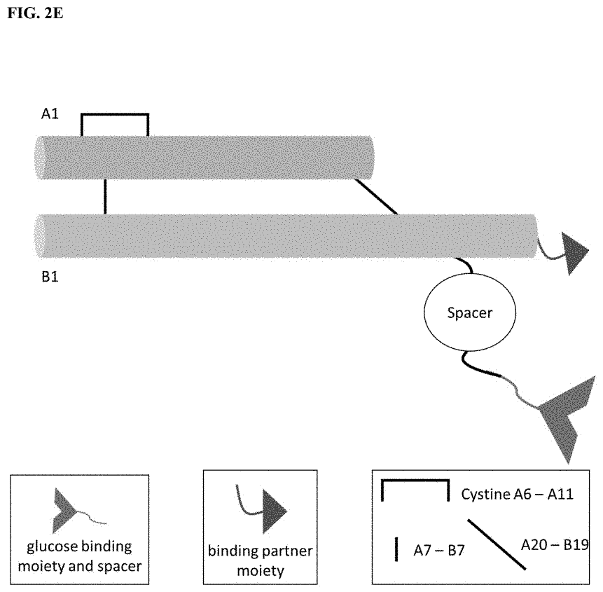

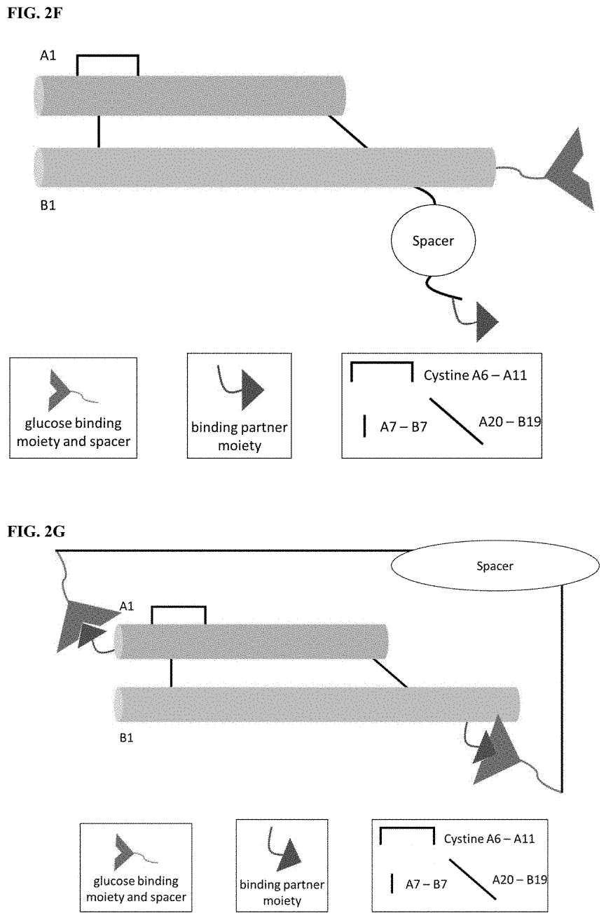

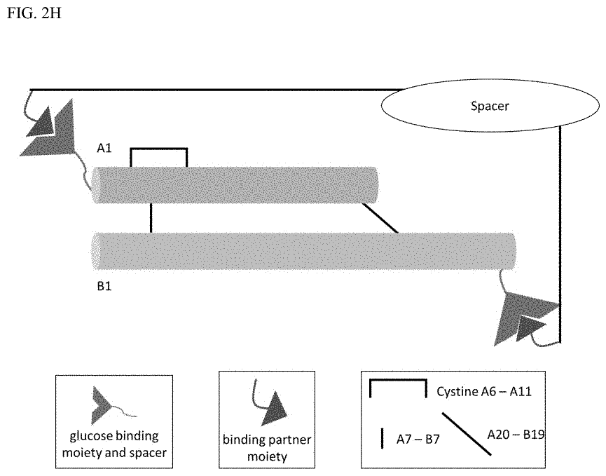

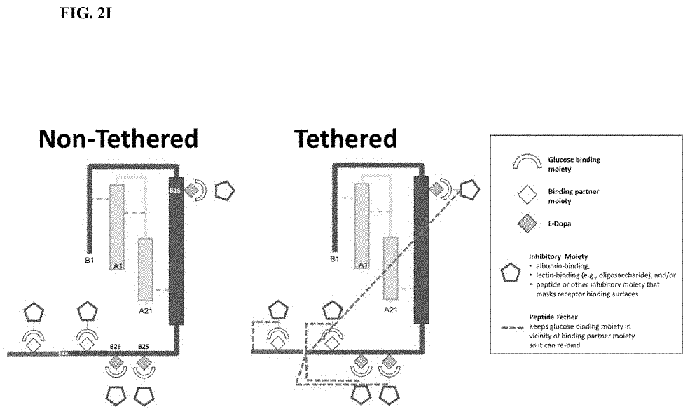

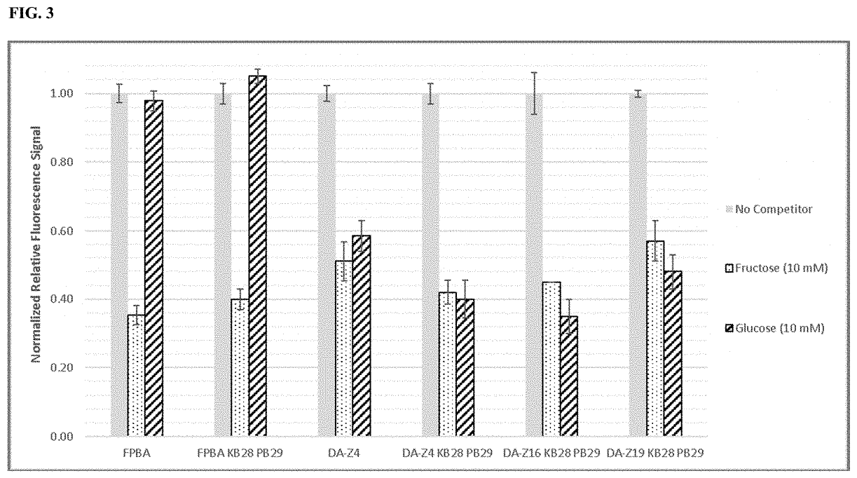

In other embodiments, the glucose-binding moiety is attached to a spacer extension of the C-terminus of the B-chain and the partner moiety is attached to B24, B25 or other positions on the B-chain. See FIG. 2C. Reversible interaction of the glucose-binding and binding partner moieties inhibits binding to the insulin receptor due to the steric bulk of the spacer element, constrained conformation, or other mechanism. Examples of spacer elements include, but are not limited to, PEG, peptidic and hydrocarbon spacers as described. In other embodiments the glucose-binding moiety is attached to B24, B25 or other positions on the B-chain and the binding partner moiety is attached to a spacer extension of the C-terminus of the B-chain. See FIG. 2D. Exemplary “fold-back” structures in accordance with these embodiments (as illustrated in FIGS. 2C and 2D) are shown with the indicated sensors in DA-Z19-1, DA-Z19-m, DA-Z19-p, and DA-Z19-q in Table 1.

In another embodiment the glucose-binding moiety is attached to B24, B25 or other positions on the B-chain through a spacer extension and the partner moiety is attached to the C-terminus of the B-chain. See FIG. 2E. Reversible interaction of the glucose-binding and binding partner moieties inhibits binding to the insulin receptor due to the steric bulk of the spacer element, constrained conformation, or other mechanism. Examples of spacer elements include, but are not limited to, PEG, peptidic and hydrocarbon spacers as described. In other embodiments the binding partner moiety is attached to B24, B25 or other positions on the B-chain through a spacer extension and the glucose binding moiety is attached to the C-terminus of the B-chain. See FIG. 2F.

Thus, in some embodiments, the insulin analogue comprises an A-chain polypeptide and a B-chain polypeptide and where: the A-chain is modified with a glucose-binding moiety and binding partner moiety each coupled to the A-chain through a spacer, or the B-chain is modified with a glucose-binding moiety and a binding partner moiety each coupled to the B-chain through a spacer. In some such embodiments, the glucose-binding moiety and binding partner moiety are attached at or near the N-terminus of the A-chain; or the glucose-binding moiety and the binding partner moiety are attached at or near C-terminus of the B-chain. For example, the glucose-binding moiety and the binding partner moiety may be each attached to a position selected from A0, A1-A10. Alternatively, the glucose-binding moiety and the binding partner moiety are each attached to a position selected from B16-B30. In still other embodiments, a glucose binding moiety or binding partner moiety is linked to a B-chain extension, wherein the extension has additional amino acid positions B31, B32, B33, B34 or further extended. The amino acid at position B31 may be cysteine or a homocysteine and the amino acid at position B32 may be cysteine or a homocysteine.

In other embodiments, the glucose-binding moiety or binding partner moiety is not connected directly to insulin, but is part of a “clamp” structure. A “clamp” can be defined as a spacer molecule flanked by a set of two moieties, glucose binding or binding partner. The moieties on the clamp bind to their complementary moieties which are in turn connected to the insulin analogue. See FIGS. 2G and 2H. Examples of spacer elements include, but are not limited to, PEG, peptidic spacers, and hydrocarbon linkers, as described above.

Thus, in some embodiments, the insulin analogue or complex comprises an A-chain polypeptide and a B-chain polypeptide and where: the A-chain is modified with a glucose-binding moiety or binding partner moiety, and the B-chain is modified with a glucose-binding moiety or binding partner moiety, and wherein the glucose-binding moiety or binding partner moiety of the A chain and the glucose-binding moiety or binding partner moiety of the B chain are bound by cognate binding partners attached through a linker. See FIGS. 2G and 2H.

In some embodiments, the glucose binding moiety or binding partner moiety is bound to an inhibitory moiety. The inhibitory moiety is of sufficient size to inhibit binding of the insulin analogue to its receptor. When glucose is present in sufficient concentrations, the glucose binding moiety binds to glucose and releases the active insulin analogue, separating it from the inhibitory moiety. For example, the inhibitory moiety could be an albumin binder that consequently binds the glucose binding/partner moiety, and therefore the insulin analogue, to albumin. In other examples the inhibitory moiety could be lectin-binding, or a peptidic or other moiety that masks receptor binding surfaces. The inhibitory moiety bound to the glucose binding moiety or partner moiety may optionally be attached to the insulin analogue via a tether element to keep the inhibitory moiety and glucose binding moiety near the partner moiety (or vice versa) to make the reaction reversible. In this embodiment, the tether element must be attached to the insulin analogue in such a way as to allow the insulin analogue to become active when the glucose binding moiety is bound to glucose. The tether element can be but is not limited to an amide linker, an acyl linker (e.g., a hydrocarbon linker), a peptidic linker or a PEG linker. See FIG. 2I.

Thus, in some embodiments, the invention provides an insulin analogue that comprises an A-chain polypeptide and a B-chain polypeptide, and where the A-chain and/or the B chain is modified with a glucose-binding moiety or a binding partner moiety, and the cognate binding partner is bound to the glucose-binding moiety or binding partner moiety so as to inhibit binding of the insulin analogue to the insulin receptor. The cognate binding partner can be conjugated to a macromolecular carrier optionally through a spacer. Exemplary macromolecular carriers include albumin or an albumin-binding molecule. Other macromolecular carriers can be used, as long as they are able to sterically hinder binding of the insulin analogue to the insulin receptor.

In some embodiments, a glucose-binding moiety or a binding partner moiety are attached at or near the N-terminus of the A-chain (which can be an extended A chain); and/or a glucose-binding moiety and/or a binding partner moiety are attached at or near C-terminus of the B-chain (which can be an extended B chain). For example, in some embodiments, a glucose-binding moiety or a binding partner moiety are attached to a position selected from A1-A10 or at A0 or longer N-terminal extension. In some embodiments, a glucose-binding moiety or a binding partner moiety are attached to a position selected from B16-B30. In some embodiments, a glucose-binding moiety or binding partner moiety is linked to a B-chain extension, such as an extension with an additional amino positions B31, B32, B33, B34 or further extended. In some embodiments, the amino acid at position B31 is a cysteine or a homocysteine and the amino acid at position B32 is cysteine or a homocysteine.

In various embodiments, the present invention contemplates the use of insulin analogues that have one or more mutations. The mutation(s) may be (independently) a natural or non-natural (e.g., non-genetically encoded or non-standard) amino acid substitutions, insertions, or deletions.

The amino-acid sequence of human proinsulin is shown below:

Phe-Val-Asn-Gln-His-Leu-Cys-Gly-Ser-His-Leu-Val-Glu-Ala-Leu-Tyr-Leu-Val-Cys-Gly-Glu-Arg-Gly-Phe-Phe-Tyr-Thr-Pro-Lys-Thr-Arg-Arg-Glu-Ala-Glu-Asp-Leu-Gln-Val-Gly-Gln-Val-Glu-Leu-Gly-Gly-Gly-Pro-Gly-Ala-Gly-Ser-Leu-Gln-Pro-Leu-Ala-Leu-Glu-Gly-Ser-Leu-Gln-Lys-Arg-Gly-Ile-Val-Glu-Gln-Cys-Cys-Thr-Ser-Ile-Cys-Ser-Leu-Tyr-Gln-Leu-Glu-Asn-Tyr-Cys-Asn (SEQ ID NO:1)

The amino-acid sequence of the A-chain of human insulin is shown below:

### Gly-Ile-Val-Glu-Gln-Cys-Cys-Thr-Ser-Ile-Cys-Ser-Leu-Tyr-Gln-Leu-Glu-Asn-Tyr-Cys-Asn (SEQ ID NO:2)

The amino-acid sequence of the B-chain of human insulin is shown below:

### Phe-Val-Asn-Gln-His-Leu-Cys-Gly-Ser-His-Leu-Val-Glu-Ala-Leu-Tyr-Leu-Val-Cys-Gly-Glu-Arg-Gly-Phe-Phe-Tyr-Thr-Pro-Lys-Thr (SEQ ID NO:3).

It is contemplated that the mutations can be introduced into the structure or sequence of any of the existing insulin or insulin analogues. For example, the mutations can be introduced into insulin analogues such as insulin lispro (sold under the name Humalog®), insulin aspart (sold under the name Novolog), insulin glulisine (sold under the name Apidra®), or other known insulins, including native insulin (e.g., native human insulin). It is also envisioned that insulin analogues may be made with A- and B-chain sequences derived from animal insulins, such as porcine, bovine, equine, and canine insulins. Various insulin molecules or analogues that may be used in the present invention are described in U. S Patent Publication No. 20180057559 and International Patent Publication No. WO 2017/070617, both of which are hereby incorporated by reference in their entirety.

In certain embodiments, the mutations include amino acid substitutions such as conservative amino acid substitutions, and/or non-conservative substitutions. “Conservative substitutions” include those substitutions made within a group of amino acids with similar side chains, for example: the neutral and hydrophobic amino acids glycine (Gly or G), alanine (Ala or A), valine (Val or V), leucine (Leu or L), isoleucine (Ile or I), proline (Pro or P), tryptophan (Trp or W), phenylalanine (Phe or F) and methionine (Met or M); the neutral polar amino acids serine (Ser or S), threonine (Thr or T), tyrosine (Tyr or Y), cysteine (Cys or C), glutamine (Glu or Q), and asparagine (Asn or N); basic amino acids lysine (Lys or K), arginine (Arg or R) and histidine (His or H); and acidic amino acids aspartic acid (Asp or D) and glutamic acid (Glu or E). Further, standard amino acids may also be substituted by non-standard amino acids, for example, those belonging to the same chemical class. By way of non-limiting example, the basic side chain lysine may be replaced by basic amino acids of shorter side-chain length (Ornithine, Diaminobutyric acid, or Diaminopropionic acid) or longer side-chain length (homolysine). Lysine may also be replaced by the neutral aliphatic isostere Norleucine (Nle), which may in turn be substituted by analogues containing shorter aliphatic side chains (Aminobutyric acid or Aminopropionic acid). In some embodiments, the insulin analogue has from one to five mutations with respect to the sequence of Insulin Lispro or Insulin Aspart. In some embodiments, these mutations are conservative mutations, with no more than one, two, or three non-conservative mutations or non-standard mutations.

In some embodiments, the insulin analogue may include anon-standard amino acid substitutions, e.g., insulin analogue may include non-standard amino acid substitutions selected from D-homolysine, D-lysine, D-omithine, D-diaminobutyric acid, D-diaminopropionic acid, L-homolysine L-lysine, L-omithine, L-diaminobutyric acid, or L-diaminopropionic acid. These non-standard amino acid substitutions can be made, for example, at positions A1, A4, B27, B28, B29, or B30.

In other embodiments, the insulin analogue may include a non-standard amino acid substitution at position B29. In one example, the non-standard amino acid at B29 is norleucine (Nle). In another example, the non-standard amino acid at B29 is ornithine (Orn). Insulin analogues including such non-standard amino acids are described, for example, in U.S. Patent Publication No. 2014/0303076, the entire contents of which are hereby incorporated by reference.

The insulin analogue may contain other modifications. In various embodiments, the insulin analogue may include one or more mutations at positions corresponding to the following positions of native human insulin: A3, A8, A10, A12, A13, A14, A17, and A21 of the A-chain and B2, B3, B4, B10, B13, B17, B28, and B29 of the B-chain. In some embodiments, the insulin analogue contains a substitution of aspartic acid (Asp or D) or lysine (Lys or K) for proline (Pro or P) at amino acid 28 of the B-chain (B28) or a substitution of proline for lysine at amino acid 29 of the B-chain (B29) or a combination thereof. In another example, the insulin analogue can include a substitution of lysine for asparagine at amino acid 3 of the B-chain (B3) or a substitution of glutamic acid for lysine at amino acid 29 of the B-chain (B29) or a combination thereof.

In various embodiments, the insulin analogue may include deletions of one or more amino acids. In an embodiment, the insulin analogue may include a deletion of amino acids corresponding to positions B1-B3, as described for example, in International Patent Publication No. WO2014/116753, the entire contents of which are hereby incorporated by reference. In some embodiments, the insulin analogue may include a B chain lacking amino acids B1-B3 in addition to one or more additional substitutions at A8, B24, B28, and/or B29. In an embodiment, the insulin analogue includes a B chain lacking amino acids B1-B3 and an ornithine or glutamic acid at B29.

In various embodiments, the insulin analogue may include insertions of one or more amino acids. In an embodiment, the insertions are at the C-terminus. For example, the insulin analogue may include an addition of at least two amino acids to the carboxyl end of the B-chain. In an embodiment, the B-chain includes a cysteine, homocysteine, glutamic acid or aspartic acid insertion at position B31 and an additional insertion selected from cysteine, homocysteine, glutamic acid, alanine, and aspartic acid at position B32. Such insulin analogues are described, for example, in U.S. Pat. No. 8,399,407, the entire contents of which are hereby incorporated by reference.

In one aspect, the present invention is related to a composition comprising one or more insulin analogues disclosed herein and a pharmaceutically acceptable carrier or excipient. In various embodiments, the pharmaceutical composition includes one or more of a pharmaceutically acceptable buffer, stabilizing agent, surfactant, solubilizing agent, charge-masking agent, anti-aggregation agent, diffusion-enhancing agent, absorption enhancing agent, and preservative. These agents can be used in combination and function synergistically to, for example, enhance insulin absorption, promote a more rapid insulin pharmacokinetics, and increase insulin stability.

The insulin compositions of the present invention can be formulated into any suitable form appropriate for the desired use and route of administration. For example, the pharmaceutical composition can take the form of solutions, suspensions, emulsion, drops, tablets, pills, pellets, capsules, powders, aerosols, sprays, or any other form suitable for administration of insulin. In some embodiments, the compositions of the present invention are formulated for administration parenterally, intradermally, intramuscularly, intranasally, sublingually, via pulmonary route, or via oral administration. In one embodiment, the insulin composition is formulated for subcutaneous administration.

Another aspect of the present invention is related to a method of lowering blood sugar of a patient comprising administering a physiologically effective amount of the insulin analogue as disclosed herein or a composition thereof.

The present invention is further illustrated by the following non-limiting examples.

### Example 1: Synthesis of Methyl (S)-2,3-diaminopropanoate

Methanol (50 mL) and (S)-2,3-diaminopropanoic acid (2.0 g, 19.2 mmol) were added to a 100 mL round bottom flask equipped with a condenser and a magnetic stirring bar. This solution was vigorously stirred at room temperature and 1 mL of sulfuric acid was added to this solution.

The solution was heated to 90° C. and allowed to reflux for 18 hours. Methanol was removed in vacuo and the solids obtained after the removal of methanol were dissolved with ethyl acetate and washed with water. The organic layer obtained after washing was dried over sodium sulfate. Subsequently, ethyl acetate was removed in vacuo to obtain the product as a yellow oil. The yield obtained from the synthesis was 2.1 g (i.e., 91%). The reaction scheme is shown below:

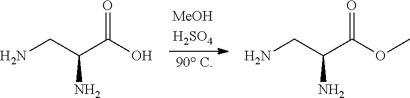

### Example 2: Synthesis of mDA-Z4 (methyl (S)-2,3-bis(1-hydroxy-1,3-dihydrobenzo[c][1,2]oxaborole-6-carboxamido)propanoate)

N,N′-dimethylformamide (10 mL) was added along with 150 mg of methyl (S)-2,3-diaminopropanoate (1.26 mmol) to a 100 mL round bottom flask containing a magnetic stir-bar. 1-hydroxy-1,3-dihydrobenzo[c][1,2]oxaborole-6-carboxylic acid (1.36 g, 7.8 mmol) was added to the round bottom flask followed by 1-ethy-3-(3-dimethylaminopropyl)carbodiimide (440 mg, 2.65 mmol) and 4-dimethylaminopyridine (324 mg, 2.65 mmol).

This solution was stirred overnight, the solvent was removed in vacuo, and the solids obtained after the removal of the solvent were dissolved in ethyl acetate. The organic layer was extracted twice with water and, subsequently, the organic layer was removed to reveal a yellow oil (298 mg, 0.776 mmol, 54% yield). The reaction scheme is shown below:

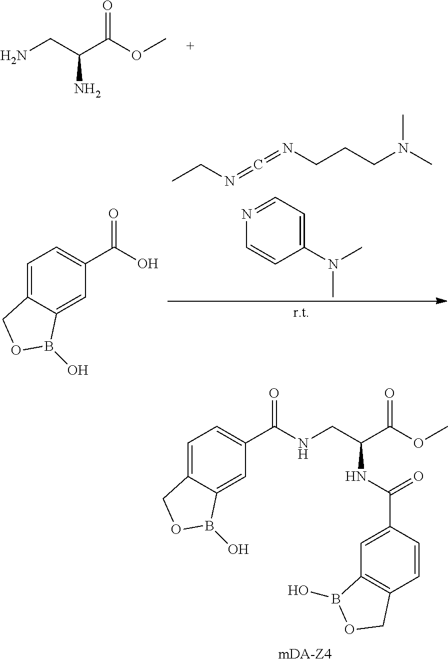

### Example 3: Synthesis of DA-Z4 (S)-2,3-bis(1-hydroxy-1,3-dihydrobenzo[c][1,2]oxaborole-6-carboxamido)propanoic acid

Potassium hydroxide (25%, v/v) was added to mD-Z4 (298 mg, 0.776 mmol) in a 100 mL round bottom flask containing a stir bar. This solution was allowed to stir at room temperature for one hour. The flask was then submerged in an ice bath and concentrated hydrochloric acid was added. Upon adjustment to a pH<1 a white precipitate formed. This white precipitate was isolated via filtration to yield 263 mg of D-Z5 (MS: [MH+]=437.3). The reaction scheme is shown below:

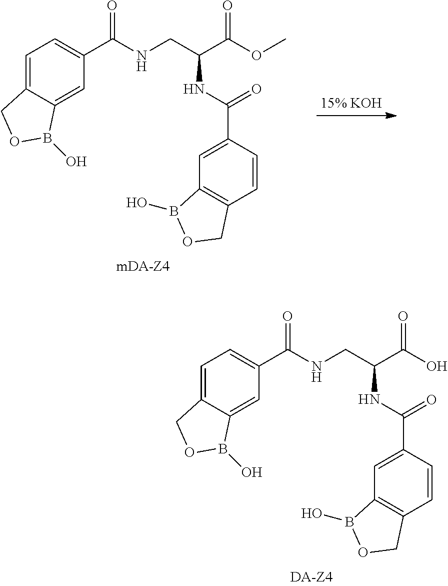 [SMILES: COC(=O)[C@H](CNC(=O)c1ccc2c(c1)B(O)OC2)NC(=O)c1ccc2c(c1)B(O)OC2 ; O=C(NC[C@H](NC(=O)c1ccc2c(c1)B(O)OC2)C(=O)O)c1ccc2c(c1)B(O)OC2]

### Example 4: Synthesis of DA-Z4 KB28 PB29

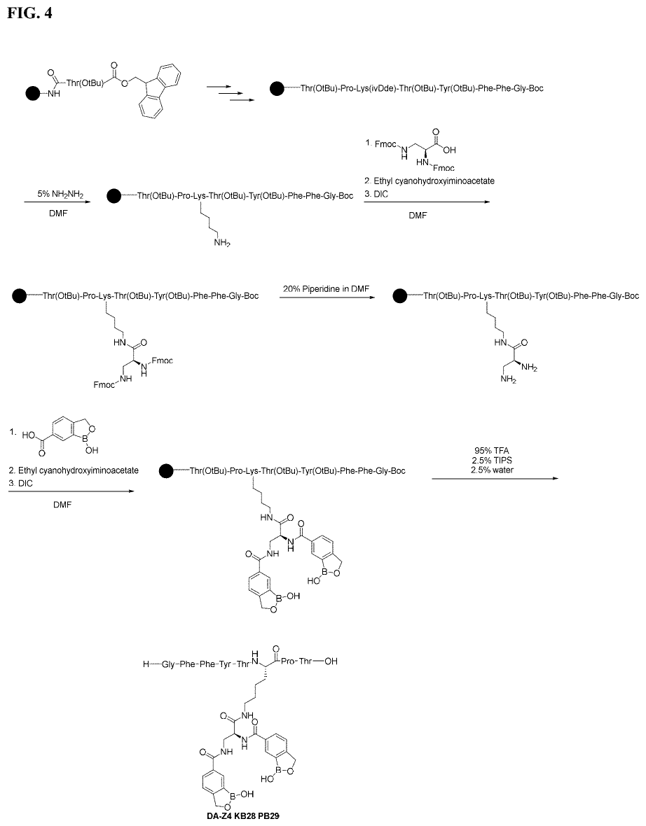

A 25-mL peptide synthesis flask charged with 500 mg (0.275 mmol) of Fmoc-Thr(OtBu)-Wang resin was allowed to swell with dichloromethane (15 mL). The dichloromethane was removed and to this was added 25% piperidine in N′N-dimethylformamide. The flask containing the resin was allowed to agitate at room temperature for 25 minutes. The solution was drained, and the resin was subsequently washed with DMF, DCM, MeOH, DCM, and DMF (15 mL). The desired peptidic sequence was achieved using the appropriate amino acid (1.1 mmol) Oxyma Pure (ethyl cyano(hydroximino)acetate) (1.1 mmol) and N, N′-diisopropylcarbodiimide (DIC) (1.1 mmol) as coupling reagents dissolved in DMF (15 mL). The flask was agitated at room temperature for 45 minutes. After successful coupling of each amino acid, the resin was washed. Fmoc protecting groups were removed from each residue with 25% piperidine in DMF (15 mL). After the full sequence was obtained, the ivDde (4,4-dimethyl-2,6-dioxocyclohex-1-ylidene)-3-methylbutyl) protecting group was removed using 5% hydrazine hydrate (NH₂NH₂.xH₂O) in DMF (v/v, 15 mL). The resin was treated for 45 minutes with this solution a total of three times. After treatment, the resin was washed with DMF, DCM, MeOH, DCM, and DMF (15 mL). (S)-2,3-bis((((9H-fluoren-9-yl)methoxy)carbonyl)amino)propanoic acid was attached using Oxyma Pure (1.1 mmol) and DIC (1.1 mmol) dissolved in DMF (15 mL). The flask was agitated at room temperature for 60 minutes. The resin was washed with DMF, DCM, MeOH, DCM, and DMF (15 mL). The Fmoc protecting groups were removed with 25% piperidine in DMF (25 mins, 15 mL). The resin was washed with DMF, DCM, MeOH, DCM, and DMF (15 mL). Next 1-hydroxy-1,3-dihydro-2,1-benzoxaborole-6-carboxylic acid (1.65 mmol) was conjugated to the free amines using Oxyma Pure (1.65 mmol) and DIC (1.65 mmol). This was allowed to react at room temperature for 180 minutes. The solution was drained, and the resin was washed with DMF, DCM, MeOH, DCM, and DMF (15 mL). The resin was treated with a trifluoroacetic acid (TFA) solution (95% TFA, 2.5% triisopropylsilane, and 2.5% water) for 120 mins. The solution was drained into a round bottom flask. The reaction solution was concentrated by removing TFA in vacuo. The residue was then transferred to a 50-mL conical tube and triturated with cold diethyl ether. The solid were collected via centrifugation. The solids were washed once more with cold diethyl ether. Residual diethyl ether was removed under vacuum to obtain a solid. This solid was dissolved in water and purified to homogeneity by reverse phase HPLC with an eluent of solvent A (water/0.1% TFA) and solvent B (ACN/0.1% TFA). The purified DA-Z4 KB28 PB29 was characterized by LC/MS. Obtained: 1366.92 [M+H]. The scheme is illustrated in FIG. 4.

### Example 5: Alizarin Red S Assay

Stock solutions of test compounds, competitor (polyols, i.e., fructose and glucose), and Alizarin Red S (ARS) were dissolved in 0.1 mM Sodium Phosphate, pH 7.4. In a 96-well plate, stock solutions were combined to final concentrations of 20 μm ARS, 0.1 mM test compound, and varying competitor concentrations (0, 5, 10, 15, and/or 25 mM). Final solution (75 μL) was added to each plate well. Fluorescence data was acquired on Molecular Devices SpectraMax M2 with excitation at 456 nm and emission at 600 nm.

In this assay, the selectivity of synthesized constructs for glucose was determined using the ARS competitive assay. Test articles were incubated with ARS and competitors (i.e. glucose and fructose) were added at varying concentrations. ARS fluorescence increases when the molecule interacts with boronic acids. The fluorescence was measured after the addition of the competitor. A decrease in fluorescence indicates the displacement of the ARS molecule by the sensor. All data were normalized to the fluorescence of an untreated solution (ARS bound to the sensing unit). When a solution of fluoro-phenylboronic acid (FPBA) is treated with fructose, the fluorescence signal decreases suggesting a strong interaction between the two molecules. This was not observed when FPBA was incubated with glucose. A solution of DA-Z4 [(S)-2,3-bis(1-hydroxy-1,3-dihydrobenzo [c][1,2]oxaborole-6-carboxamido)propanoic acid] and ARS shows a similar response to both glucose and fructose suggesting that this construct does not preferentially bind to fructose over glucose. This trend was also observed for DA-Z4 KB28 PB29, DA-Z16 KB28 PB29, and DA-Z19 KB28 PB29. These are peptides modified to include glucose sensors at the lysyl residue. DA-Z4 is an L-diaminopropionic acid modified to include two benzoxoxaboroles. DA-Z16 is an D-diaminobutyric acid modified to include two benzoxaboroles. DA-Z19 is a D-diaminopropionic acid modified to include two benzoxaboroles.

The ARS data indicates that DA-Z4, DA-Z4 KB28 PB29, DA-Z16 KB28 PB29, and DA-Z19 KB28 PB29 divalent boronic acids, exhibit a stronger binding to glucose compared to monoboronic acids such as FPBA and FPBA KB28 PB29.

### Example 6: IR Activation Assay

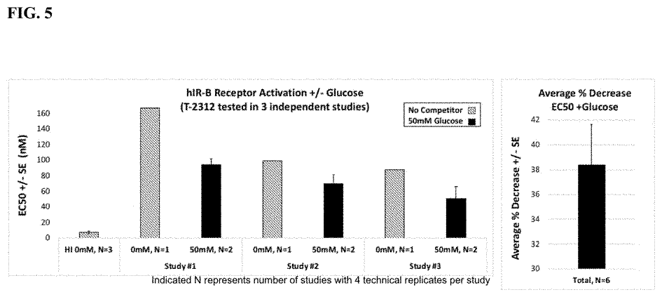

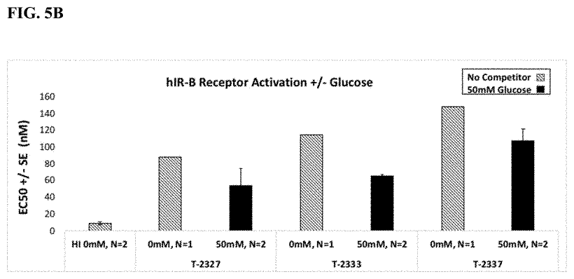

Insulin receptor activation was measured in Chinese Hamster Ovary (CHO) cells overexpressing the B-isoform of the human insulin receptor (hIR-B). Briefly, CHO-hIR-B cells seeded in 96-well plates were treated with serially diluted ligands for 20 min at 37° C. followed by fixation in formaldehyde. Fixed cells were permeabilized with detergent and total phospho-Tyrosine detected using 4G10 1° Ab and anti-mouse-IgG-800-CW 2° Ab. Data were normalized to cell count by DRAQ5 staining of DNA. EC₅₀ values were calculated from four-parameter log-logistic curve fitting of the phosphorylation signal. In FIG. 5A the ligand tested (T-2312) is comprised of a 2-chain insulin analogue conjugated with a glucose-binding moiety at LysB28, DA-Z19 in this example, and a binding partner moiety, 3,4-dihydroxy benzoic acid (DHBA) in this example, amine-linked at the A1 position. Wild-type human insulin is included as a control. T-2312 was preincubated with or without 50 mM glucose prior to cell treatment. FIG. 5A illustrates a decrease in EC₅₀ in response to glucose representing an increase in activity in three independent studies. The average EC₅₀ decrease in response to glucose is 38.4%+5.8 from three experiments each with two technical replicates (n=6).

FIG. 5B illustrates three different analogs that demonstrate response to glucose in the IR activation assay outlined above. All are 2-chain insulin analogues with a glucose-binding moiety at or near the end of the B-chain and binding partner linked to the A1 position: T-2327 (3,4-DHBA-A1, DAZ19-Ornithine-B28), T-2333 (3,4-DHBA-A1, DAZ19-Peg1Dap-B28), D-2337 (3-methoxy-4,5-DHBA-A1, DAZ4-Lysine-B28). Peg1Dap refers to a PEG monomer linkage through diaminopropionic acid.

Exemplary glucose-binding moiety (termed “sensors” in Table 1) designs according to embodiments of the invention are illustrated in the following Table 1. Points of attachment to the insulin peptide, where not indicated, are as disclosed above. Table 1 includes various Sensor-peptide designs, but it will be understood that the peptide portion is merely illustrative of the attachment to an insulin peptide.

TABLE 1
Glucose-binding Moiety Sensor Designs

| Name | Structure |
|---|---|
| DA- Z3 | 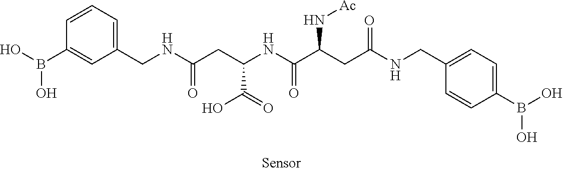 [SMILES: CC(=O)N[C@@H](CC(=O)NCc1ccc(B(O)O)cc1)C(=O)N[C@@H](CC(=O)NCc1cccc(B(O)O)c1)C(=O)O] |
| DA- Z4 | 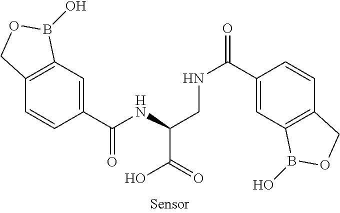 [SMILES: O=C(NC[C@H](NC(=O)c1ccc2c(c1)B(O)OC2)C(=O)O)c1ccc2c(c1)B(O)OC2] |
| DA- Z5 | 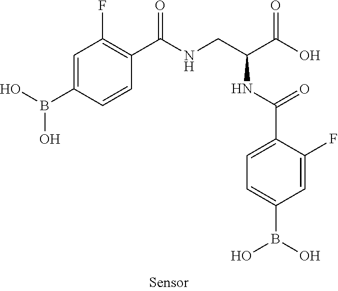 [SMILES: O=C(NC[C@H](NC(=O)c1ccc(B(O)O)cc1F)C(=O)O)c1ccc(B(O)O)cc1F] |
| DA- Z6 | 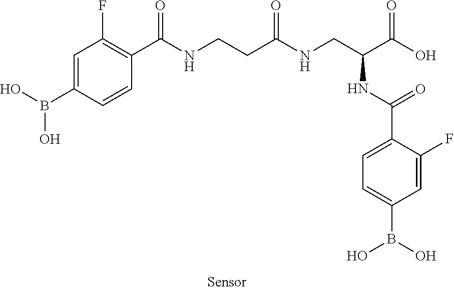 [SMILES: O=C(CCNC(=O)c1ccc(B(O)O)cc1F)NC[C@H](NC(=O)c1ccc(B(O)O)cc1F)C(=O)O] |
| DA- Z7 | 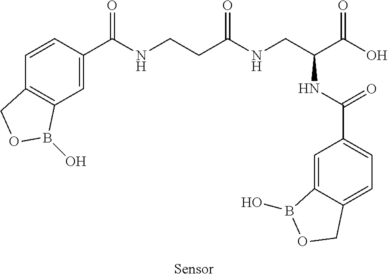 [SMILES: O=C(CCNC(=O)c1ccc2c(c1)B(O)OC2)NC[C@H](NC(=O)c1ccc2c(c1)B(O)OC2)C(=O)O] |
| DA- Z9 | 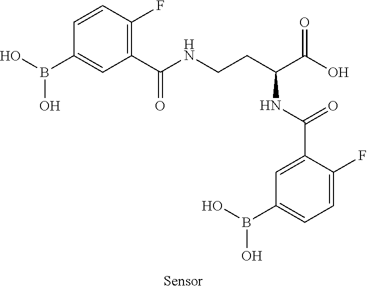 [SMILES: O=C(NCC[C@H](NC(=O)c1cc(B(O)O)ccc1F)C(=O)O)c1cc(B(O)O)ccc1F] |
| DA- Z10 | 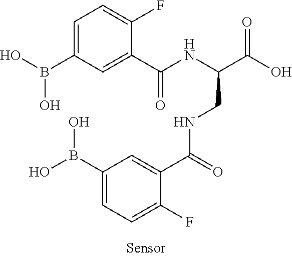 [SMILES: O=C(NC[C@@H](NC(=O)c1cc(B(O)O)ccc1F)C(=O)O)c1cc(B(O)O)ccc1F] |
| DA- Z11 | 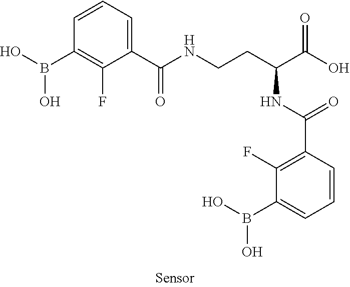 [SMILES: O=C(NCC[C@H](NC(=O)c1cccc(B(O)O)c1F)C(=O)O)c1cccc(B(O)O)c1F] |
| DA- Z12 | 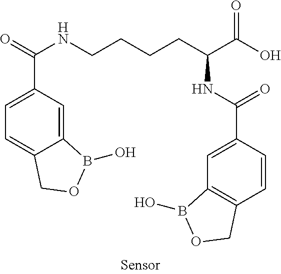 [SMILES: O=C(NCCCC[C@H](NC(=O)c1ccc2c(c1)B(O)OC2)C(=O)O)c1ccc2c(c1)B(O)OC2] |
| DA- Z13 | 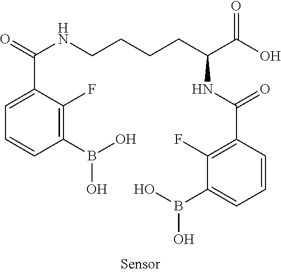 [SMILES: O=C(NCCCC[C@H](NC(=O)c1cccc(B(O)O)c1F)C(=O)O)c1cccc(B(O)O)c1F] |
| DA- Z14 | 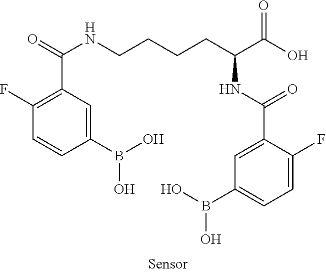 [SMILES: O=C(NCCCC[C@H](NC(=O)c1cc(B(O)O)ccc1F)C(=O)O)c1cc(B(O)O)ccc1F] |
| DA- Z15 | 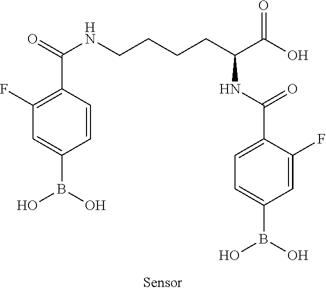 [SMILES: O=C(NCCCC[C@H](NC(=O)c1ccc(B(O)O)cc1F)C(=O)O)c1ccc(B(O)O)cc1F] |
| DA- Z17 | 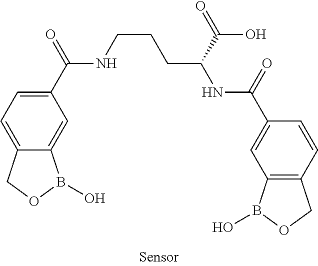 [SMILES: O=C(NCCC[C@@H](NC(=O)c1ccc2c(c1)B(O)OC2)C(=O)O)c1ccc2c(c1)B(O)OC2] |
| DA- Z18 | 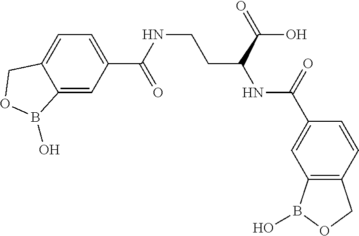 |
| DA- Z16 | 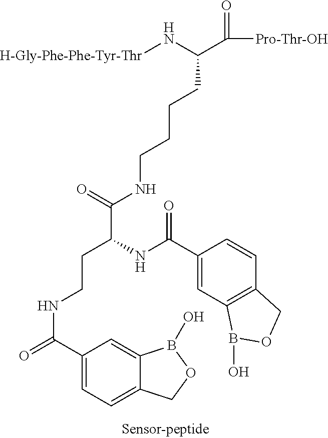 |
| DA- Z19 | 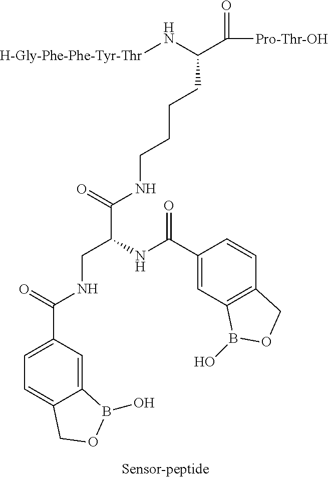 |
| DA- Z21 | 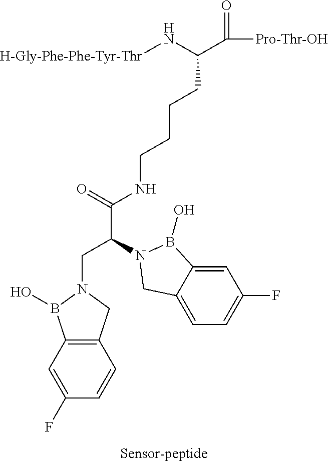 |
| DA- Z24 | 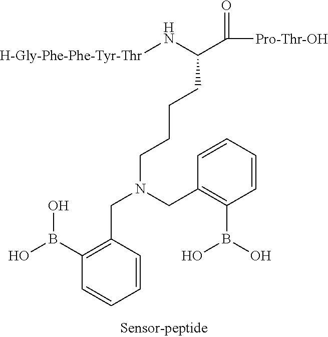 |
| DA- Z25 | 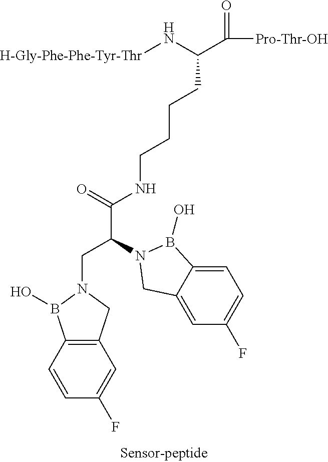 |
| DA- Z26 | 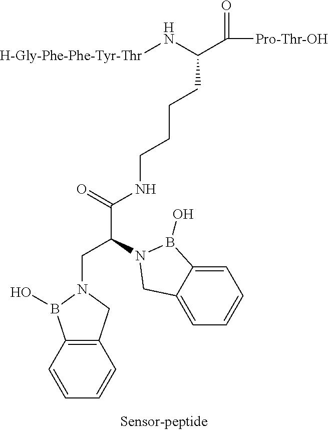 |
| DA- Z27 | 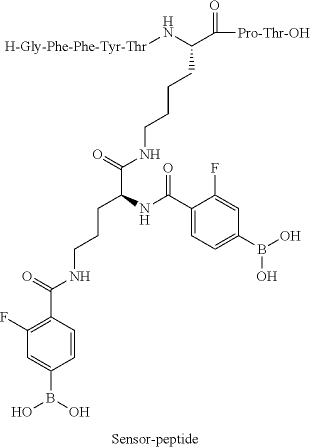 |
| DA- Z28 | 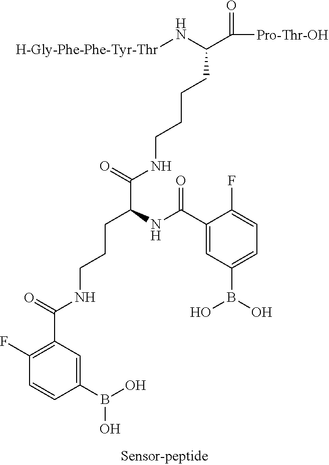 |
| DA- Z29 | 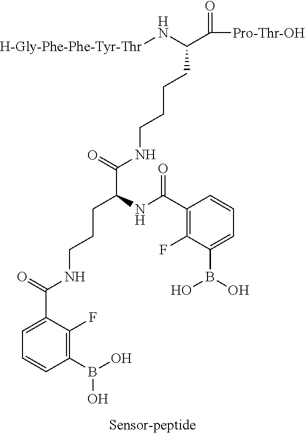 |
| DA- Z30 | 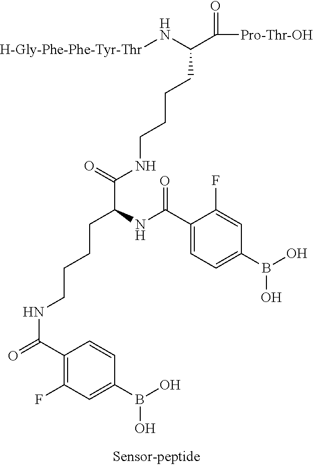 |
| DA- Z31 | 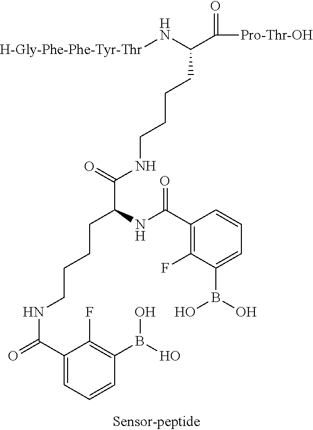 |
| DA- Z32 | 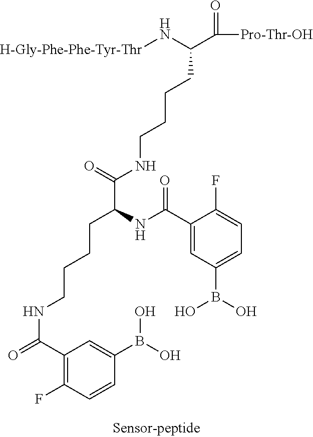 |
| DA- Z37 | 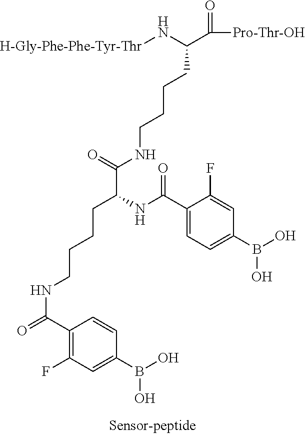 |
| DA- Z41 | 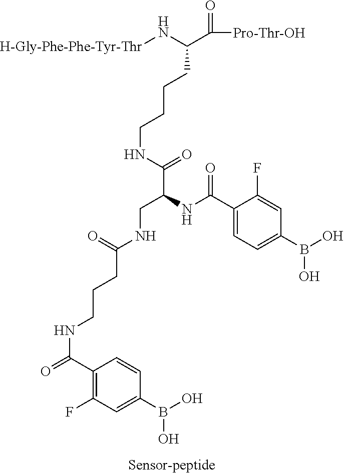 |
| DA- Z42 | 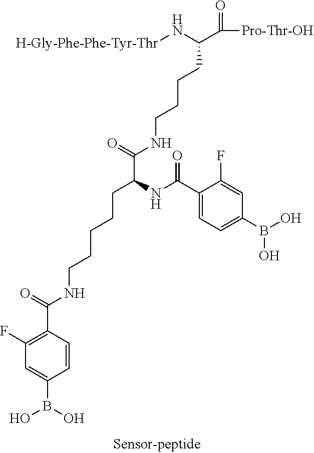 |
| DA- Z45 | 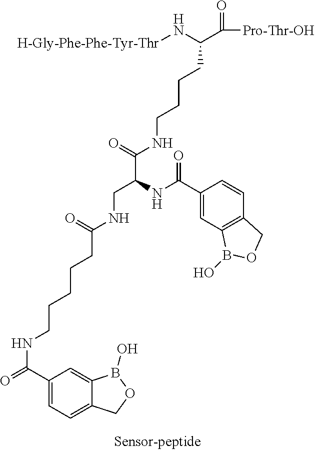 |
| DA- Z46 | 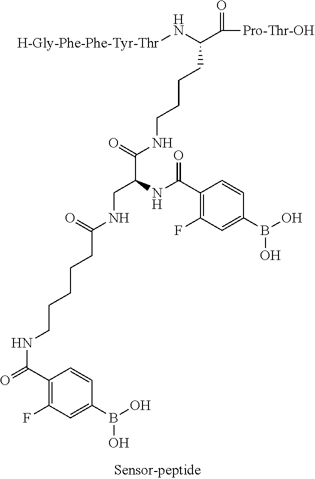 |
| DA- Z47 | 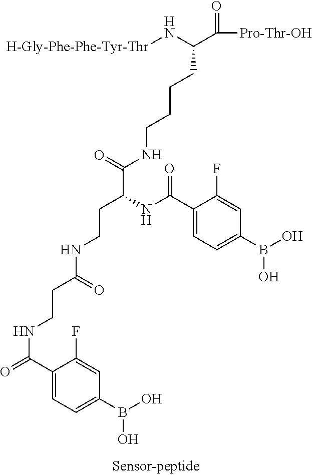 |
| DA- Z48 | 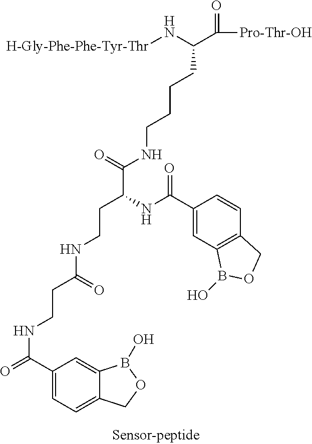 |
| DA- Z49 | 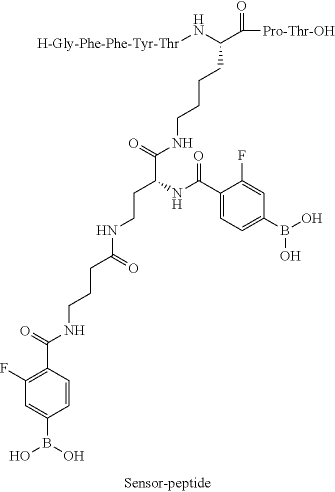 |
| DA- Z50 | 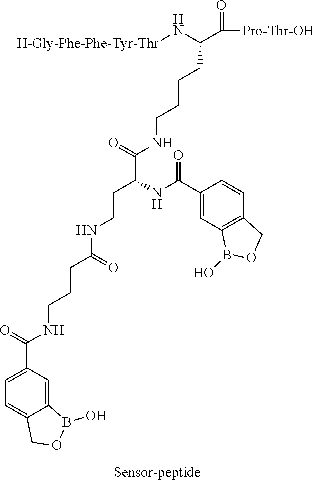 |
| DA- Z51 | 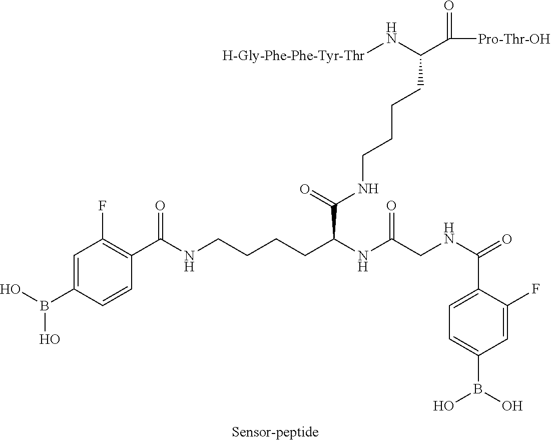 |
| DA- Z52 | 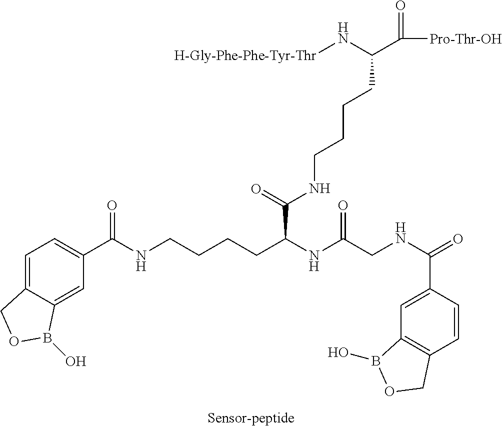 |
| DA- Z53 | 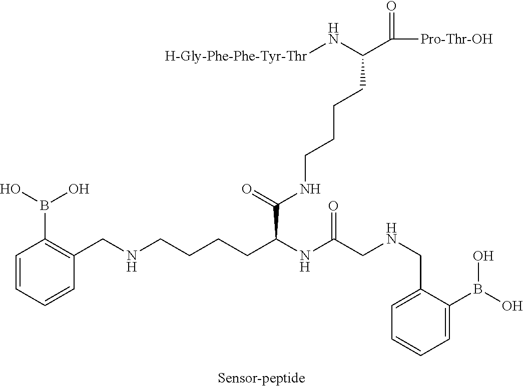 |
| DA- Z54 | 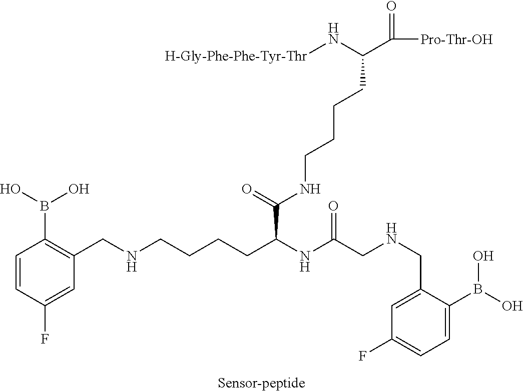 |
| DA- Z55 | 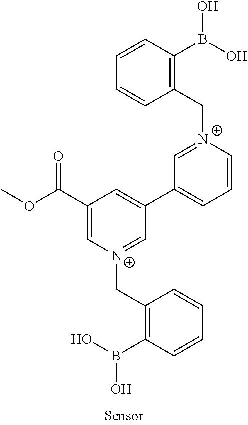 [SMILES: COC(=O)c1cc(-c2ccc[n+](Cc3ccccc3B(O)O)c2)c[n+](Cc2ccccc2B(O)O)c1] |
| DA- Z56 | 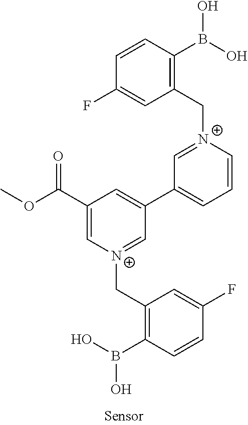 [SMILES: COC(=O)c1cc(-c2ccc[n+](Cc3cc(F)ccc3B(O)O)c2)c[n+](Cc2cc(F)ccc2B(O)O)c1] |
| DA- Z60 | 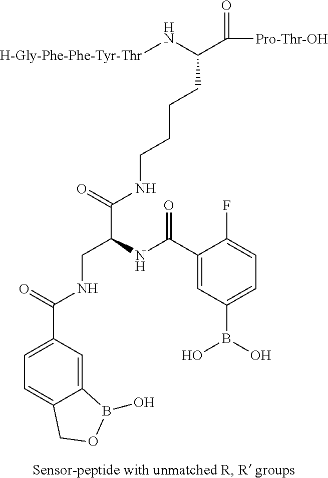 |
| DA- Z61 | Sensor peptide with unmatched R, R′ groups |
| DA- Z63 | 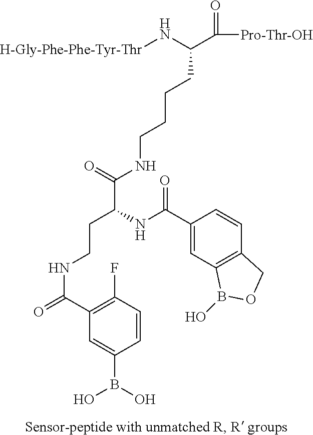 |
| DA- Z64 | 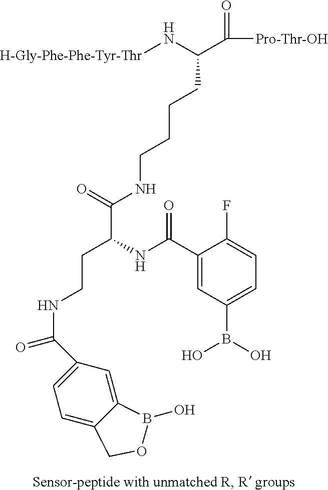 |
| DA- Z70 | 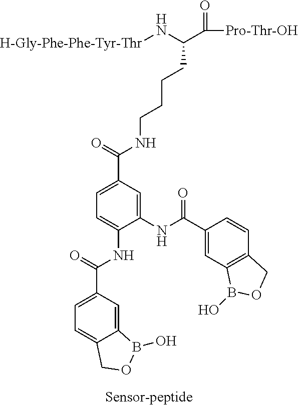 |
| DA- Z80 | 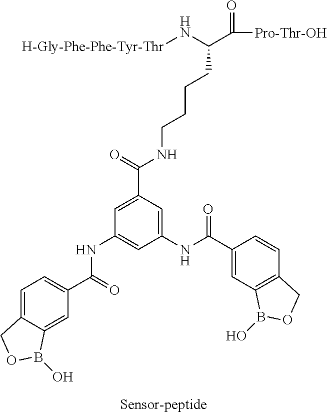 |
| DA- Z4- a | 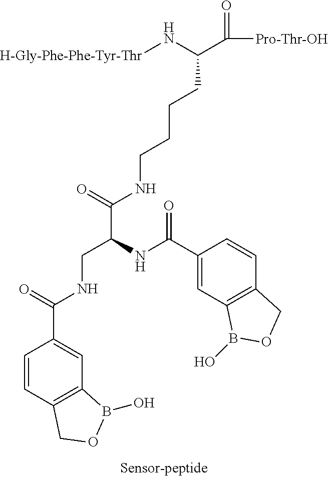 |
| DA- Z4- b | 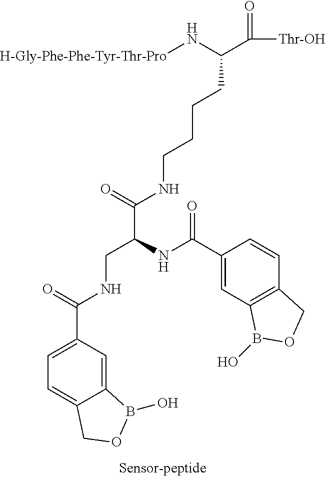 |
| DA- Z16- a | 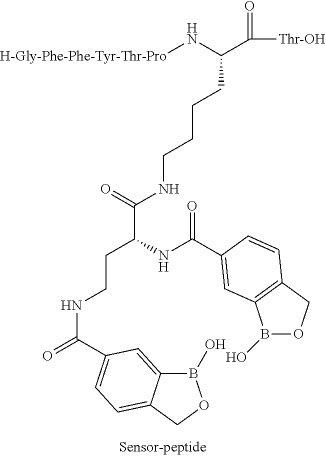 |
| DA- Z19- a | 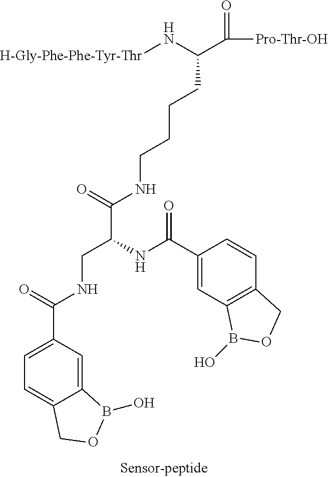 |
| DA- Z19- b | 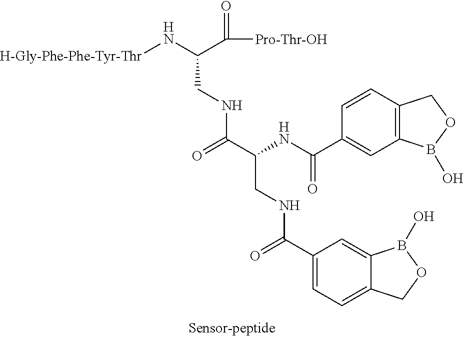 |
| DA- Z19- c | 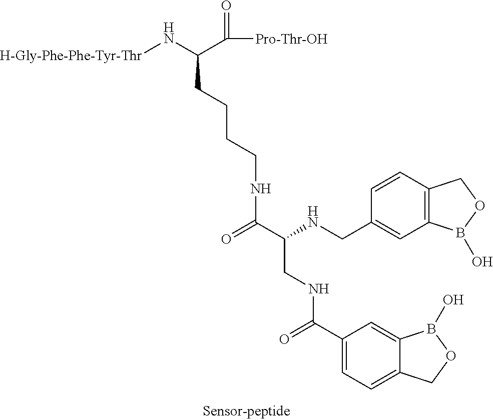 |
| DA- Z19- d | 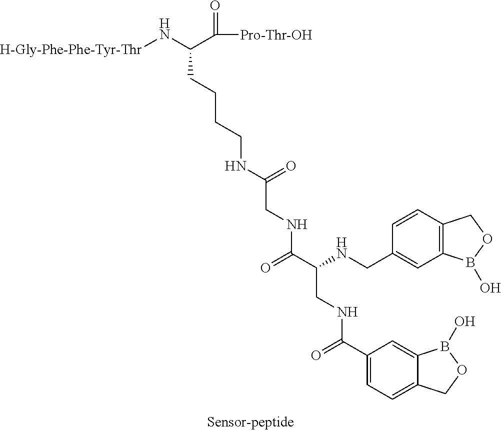 |
| DA- Z19- e | 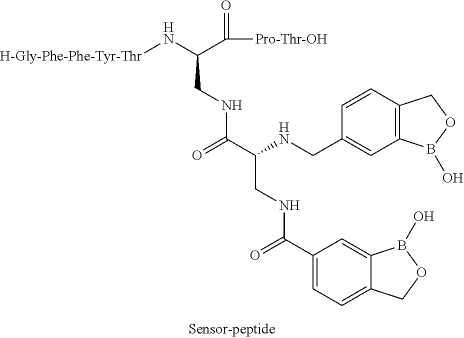 |
| DA- Z19- f | 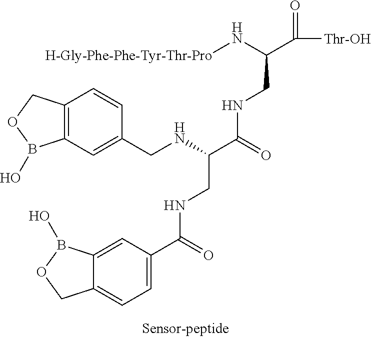 |
| DA- Z19- g | 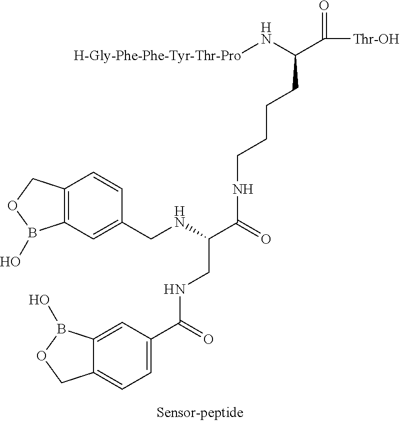 |
| DA- Z19- h | 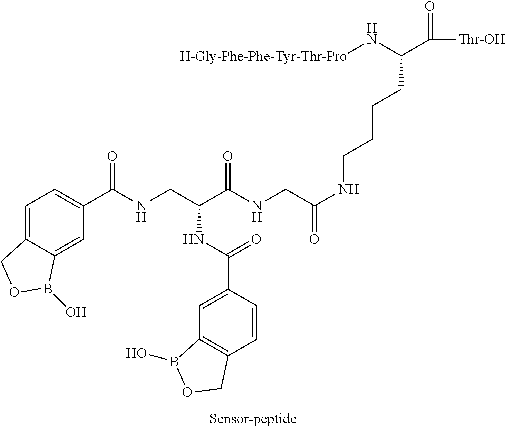 |
| DA- 19- i | 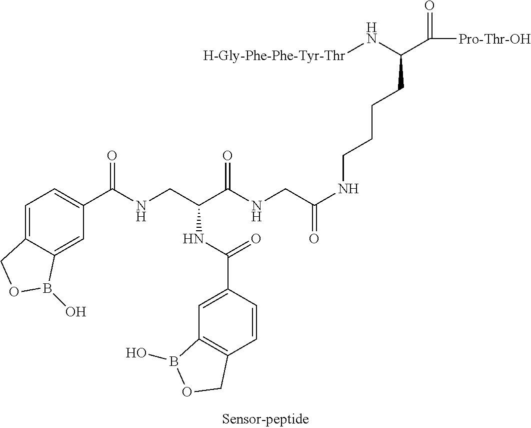 |
| DA- Z19- j | 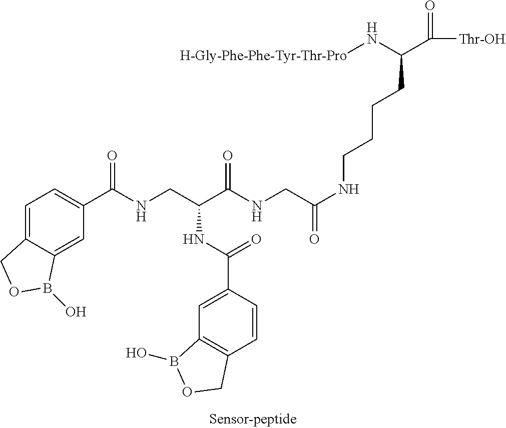 |
| DA- 19- k | 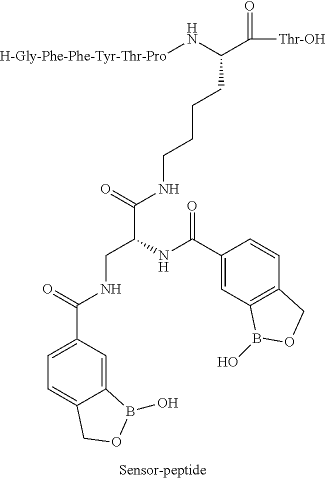 |
| DA- Z19- 1 | 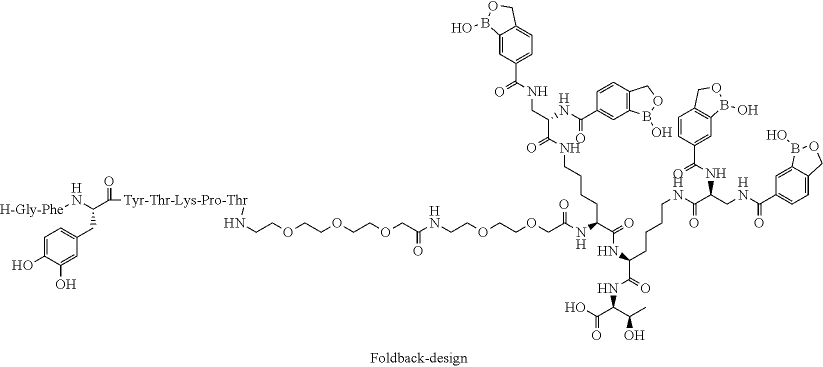 |
| DA- Z19- m | 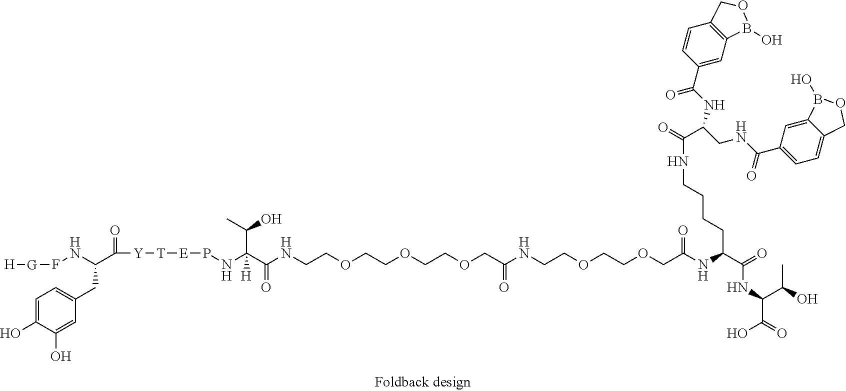 |
| DA- Z19- n | 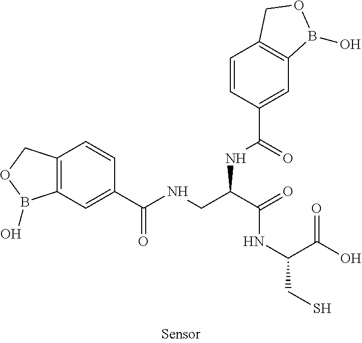 [SMILES: O=C(NC[C@@H](NC(=O)c1ccc2c(c1)B(O)OC2)C(=O)N[C@@H](CS)C(=O)O)c1ccc2c(c1)B(O)OC2] |
| DA- Z19- o | 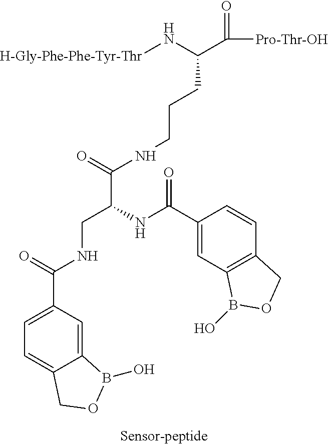 |
| DA- Z19- p | 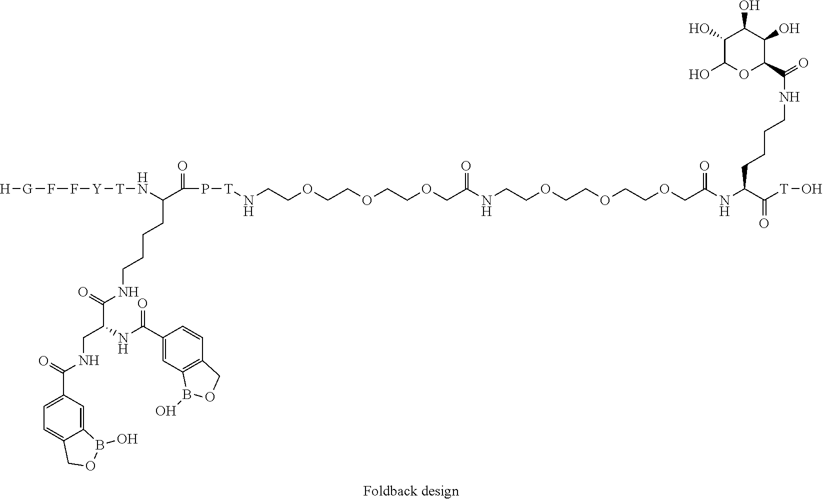 |
| DA- Z19- q | 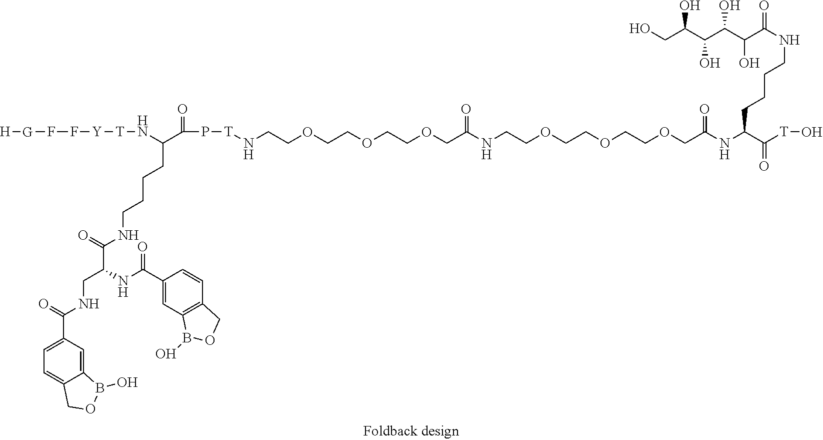 |
| DA- Z19- r | 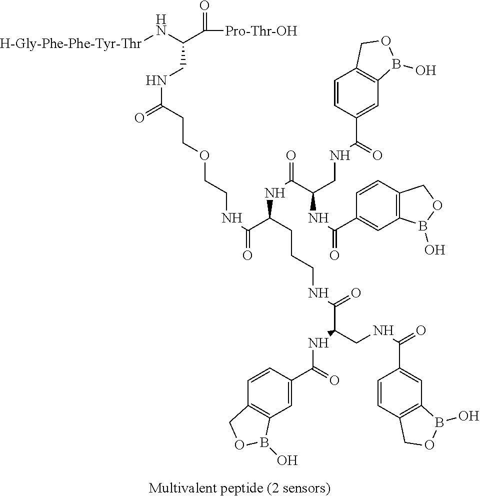 |
| DA- Z19- s | 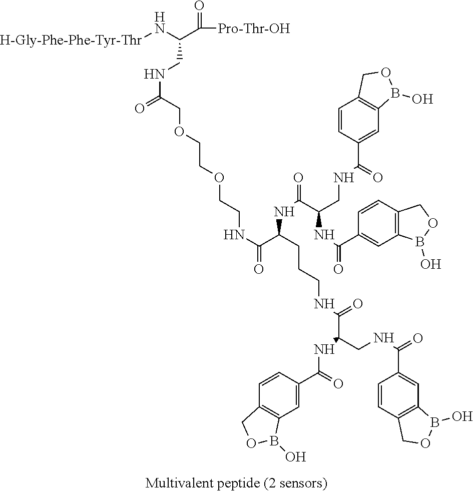 |
| DA- Z19- t | 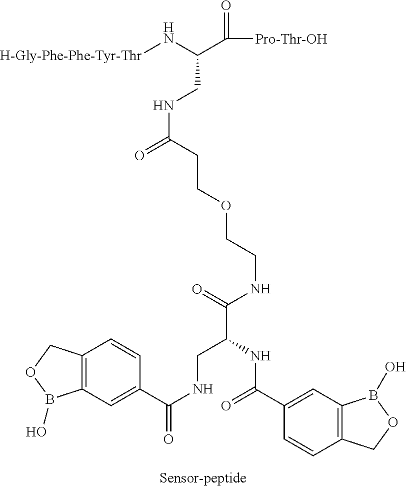 |
| DA- Z19- u | 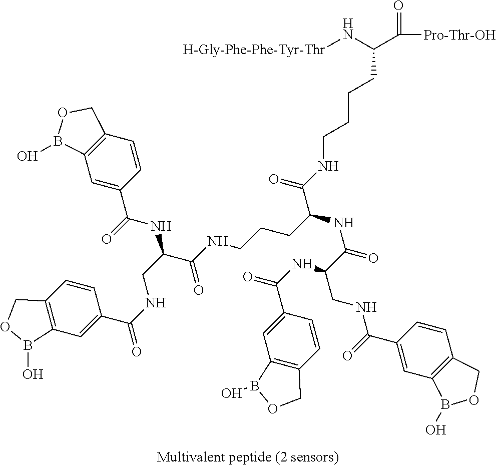 |

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

What is claimed:

1. A glucose-responsive insulin analogue or complex, comprising:
an insulin analogue,
one or more glucose-binding moieties, and
one or more binding partner moieties
wherein by interaction of the glucose-binding moiety and binding partner moiety the glucose-responsive insulin analogue or complex has low affinity for the insulin receptor at euglycemic concentrations of glucose and has increased affinity for the insulin receptor by disruption of the glucose-binding moiety and binding partner moiety interaction at hyperglycemic concentrations of glucose.

2. The insulin analogue or complex of claim 1, wherein the glucose-binding moiety is a multivalent boronic acid.

3. The insulin analogue or complex of claim 2 wherein the multivalent boronic acid moiety comprises one or more aromatic boronic acids, which is optionally a halogen-modified aromatic boronic acid.

4. The insulin analogue or complex of claim 2 or 3, wherein the multivalent boronic acid moiety is a divalent boronic acid moiety.

5. The insulin analogue or complex of claim 4, wherein the divalent boronic acid moiety further comprises a linker, which is optionally an amide linker, an acyl linker, or a PEG linker.

6. The insulin analogue or complex of claim 5, wherein the divalent boronic acid moiety is a compound of Formula (I): 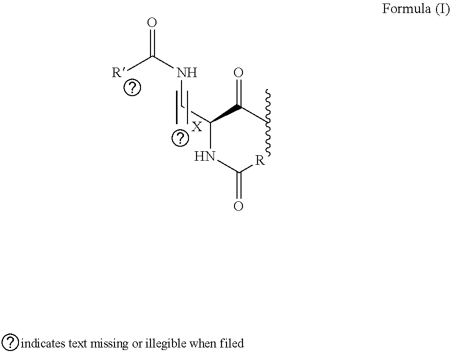
wherein:
X is 1, 2, 3, 4, or 5, and
R and/or R′ comprises a group independently selected from 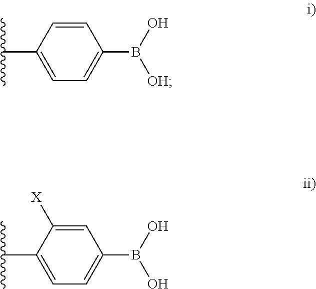
where X is selected from F, Cl, Br, I, —NH₂, —OMe, —NO₂; 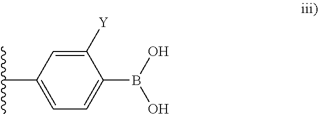
where Y is selected from F, Cl, Br, I, —NH₂, —OMe, —NO₂, —NH(CH₃), —N(CH₃)₂, —(CH₂)ₐNH(CH₃), or —N(CH₂)_{b}(CH₃)₂; a=1, 2, or 3 and b=1, 2, or 3; 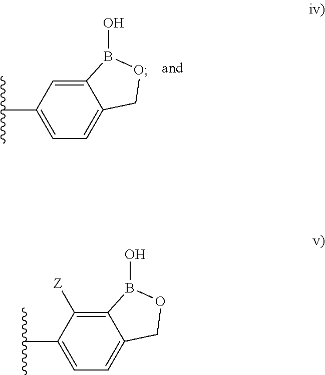
where Z is selected from F, Cl, Br, I, —NH₂, —OMe, —NO₂; and 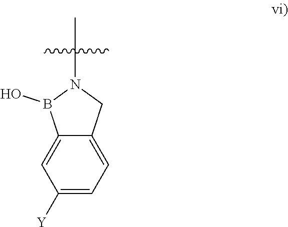
where Y is selected from F, Cl, Br, I, —NH₂, —OMe, —NO₂, —NH(CH₃), —N(CH₃)₂, —(CH₂)ₐNH(CH₃), or —N(CH₂)_{b}(CH₃)₂; a=1, 2, or 3 and b=1, 2, or 3.

7. The insulin analogue or complex of claim 3, wherein the divalent boronic acid moiety has the following structure:

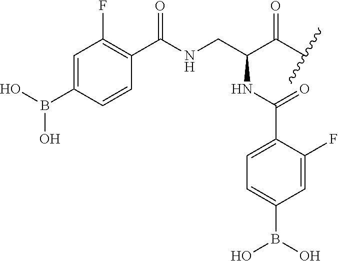 [SMILES: *C(=O)[C@H](CNC(=O)c1ccc(B(O)O)cc1F)NC(=O)c1ccc(B(O)O)cc1F]

8. The insulin analogue or complex of claim 5, wherein the divalent boronic acid moiety has the following formula:

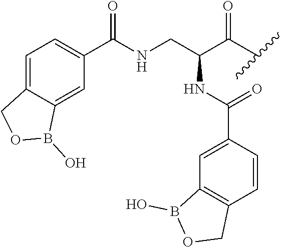 [SMILES: *C(=O)[C@H](CNC(=O)c1ccc2c(c1)B(O)OC2)NC(=O)c1ccc2c(c1)B(O)OC2]

9. The insulin analogue or complex of claim 5, wherein the divalent boronic acid moiety is a compound of Formula (II): 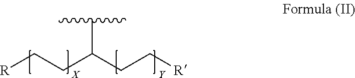
wherein:
X is 1-10, and
R and/or R′ comprises a group independently selected from 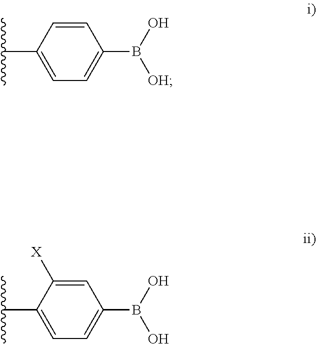
where X is selected from F, Cl, Br, I, —NH₂, —OMe, —NO₂; 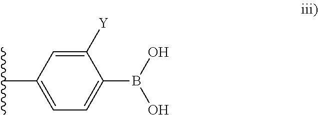
where Y is selected from F, Cl, Br, I, —NH₂, —OMe, —NO₂, —NH(CH₃), —N(CH₃)₂, —(CH₂)ₐNH(CH₃), or —N(CH₂)_{b}(CH₃)₂; a=1, 2, or 3 and b=1, 2, or 3; 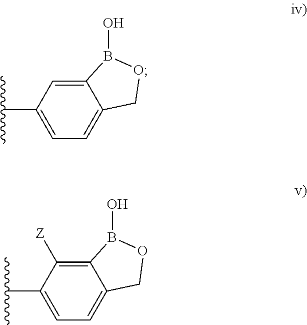
where Z is selected from F, Cl, Br, I, —NH₂, —OMe, —NO₂; and 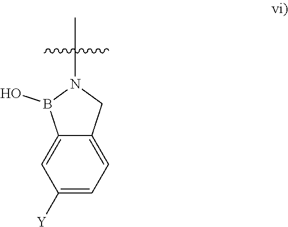
where Y is selected from F, Cl, Br, I, —NH₂, —OMe, —NO₂, —NH(CH₃), —N(CH₃)₂, —(CH₂)ₐNH(CH₃), or —N(CH₂)_{b}(CH₃)₂; a=1, 2, or 3 and b=1, 2, or 3.

10. The insulin analogue or complex of claim 5, wherein the divalent boronic acid moiety has a structure of Formula (III): 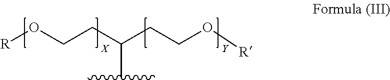
wherein:
X is 1-10, and
R and/or R′ comprises a group independently selected from 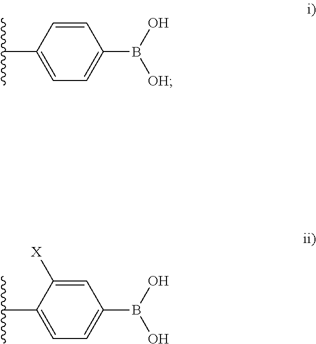
where X is selected from F, Cl, Br, I, —NH₂, —OMe, —NO₂; 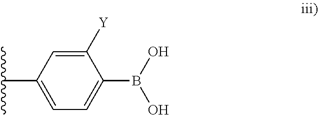
where Y is selected from F, Cl, Br, I, —NH₂, —OMe, —NO₂, —NH(CH₃), —N(CH₃)₂, —(CH₂)ₐNH(CH₃), or —N(CH₂)_{b}(CH₃)₂; a=1, 2, or 3 and b=1, 2, or 3; 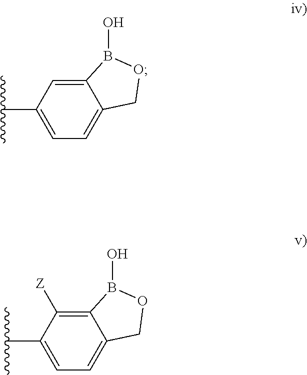
where Z is selected from F, Cl, Br, I, —NH₂, —OMe, —NO₂; and 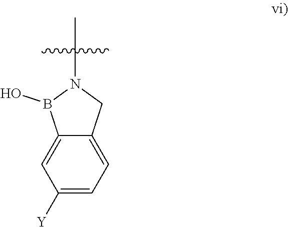
where Y is selected from F, Cl, Br, I, —NH₂, —OMe, —NO₂, —NH(CH₃), —N(CH₃)₂, —(CH₂)ₐNH(CH₃), or —N(CH₂)_{b}(CH₃)₂; a=1, 2, or 3 and b=1, 2, or 3.

11. The insulin analogue or complex of any one of claims 1 to 10, wherein the binding partner is a polyol-containing moiety.

12. The insulin analogue or complex of claim 11, wherein the polyol-containing moiety is a sugar mimetic.

13. The insulin analogue or complex of claim 11, wherein the polyol-containing moiety is natural or synthetically modified monosaccharide, disaccharide, and/or an oligosaccharide.

14. The insulin analogue or complex of any one of claims 1 to 13, wherein the glucose-binding moiety and/or the binding partner moiety are amine-linked or thiol-linked to the insulin analogue.

15. The insulin analogue or complex of claim 14, wherein the glucose binding moiety and/or the binding partner moiety are linked to an alpha-amino functional group or a side chain amino functional group of an amino acid residue of the insulin analogue.

16. The insulin analogue or complex of claim 15, wherein one or more amino acids of the insulin analogue are substituted with an amino acid selected from D-homolysine, D-lysine, D-ornithine, D-diaminobutyric acid, D-diaminopropionic acid, L-homolysine, L-lysine, L-ornithine, L-diaminobutyric acid, and L-diaminopropionic acid, which optionally are linked to the glucose-binding moiety and/or the binding partner moiety.

17. The insulin analogue or complex of any one of claims 1 to 16, wherein the glucose-binding moiety and/or the binding partner moiety is linked to the insulin analogue via a spacer.

18. The insulin analogue or complex of claim 17, wherein the spacer comprises an amide linker, an acyl linker, or a PEG linker.

19. The insulin analogue or complex of claim 18, wherein the spacer is an acyl linker with 1 to 16 carbon atoms, optionally, with one more nitrogen atoms at or near the end of the linker.

20. The insulin analogue or complex of any one of claims 1 to 19, wherein the insulin analogue comprises an A-chain polypeptide and a B-chain polypeptide and wherein:
the A-chain is modified with a glucose-binding moiety and the B-chain is modified with a binding partner moiety; or
the B-chain is modified with a glucose-binding moiety and the A-chain is modified with a binding partner moiety.

21. The insulin analogue or complex of claim 20, wherein the glucose-binding moiety is attached at or near N-terminus of the A-chain and the binding partner moiety is attached at or near C-terminus of the B-chain.

22. The insulin analogue or complex of claim 20, wherein the glucose-binding moiety is attached at or near C-terminus of the B-chain and the binding partner moiety is attached at or near N-terminus of the A-chain.

23. The insulin analogue or complex of claim 21, wherein the glucose-binding moiety is attached to a position selected from A1-A10.

24. The insulin analogue or complex of claim 23, wherein the binding partner moiety is attached to a position selected from B16-B30.

25. The insulin analogue or complex of claim 23, wherein the binding partner moiety is linked to a B-chain extension, wherein the extension has an additional B31 amino acid or additional B31 and B32 amino acids.

26. The insulin analogue or complex of claim 22, wherein the glucose-binding moiety is attached to a position selected from B16-B30.

27. The insulin analogue or complex of claim 26, wherein the binding partner moiety is attached to a position selected from A1-A10.

28. The insulin analogue or complex of claim 26, wherein the amino acid at position B31 is a cysteine or a homocysteine and the amino acid at position B32 is cysteine or a homocysteine.

29. The insulin analogue or complex of any one of claims 1 to 20 wherein the insulin analogue comprises an A-chain polypeptide and a B-chain polypeptide and wherein:
the A-chain is modified with a glucose-binding moiety or binding partner moiety, and the B-chain is modified with a glucose-binding moiety or binding partner moiety, and wherein the glucose-binding moiety or binding partner moiety of the A chain and the glucose-binding moiety or binding partner moiety of the B chain are bound by cognate binding partners attached through a linker.

30. The insulin analogue or complex of claim 29, wherein a glucose-binding moiety or binding partner moiety of the A chain is attached at or near the N-terminus of the A-chain; and the glucose-binding moiety or binding partner moiety of the B chain is attached at or near C-terminus of the B-chain.

31. The insulin analogue or complex of claim 30, wherein the glucose-binding moiety or binding partner moiety of the A chain is attached to a position selected from A1-A10.

32. The insulin analogue or complex of claim 31, wherein the glucose-binding moiety or the binding partner moiety of the B chain is attached to a position selected from B16-B30.

33. The insulin analogue or complex of any one of claims 29 to 32, wherein a glucose binding moiety or binding partner moiety is linked to a B-chain extension, wherein the extension has an additional B31 amino acid or additional B31 and B32 amino acids.

34. The insulin analogue or complex of claim 33, wherein the amino acid at position B31 is a cysteine or a homocysteine and the amino acid at position B32 is cysteine or a homocysteine.

35. The insulin analogue or complex of any one of claims 1 to 19, wherein the insulin analogue comprises an A-chain polypeptide and a B-chain polypeptide and wherein:
the A-chain is modified with a glucose-binding moiety and binding partner moiety each coupled to the A-chain through a spacer, or the B-chain is modified with a glucose-binding moiety and a binding partner moiety each coupled to the B-chain through a spacer.

36. The insulin analogue or complex of claim 35, wherein the glucose-binding moiety and binding partner moiety are attached at or near the N-terminus of the A-chain; or the glucose-binding moiety and the binding partner moiety are attached at or near C-terminus of the B-chain.

37. The insulin analogue or complex of claim 36, wherein the glucose-binding moiety and the binding partner moiety are attached to a position selected from A1-A10.

38. The insulin analogue or complex of claim 36, wherein the glucose-binding moiety and the binding partner moiety are attached to a position selected from B16-B30.

39. The insulin analogue or complex of claim 36, wherein a glucose binding moiety or binding partner moiety is linked to a B-chain extension, wherein the extension has an additional B31 amino acid or additional B31 and B32 amino acids.

40. The insulin analogue or complex of claim 39, wherein the amino acid at position B31 is a cysteine or a homocysteine and the amino acid at position B32 is cysteine or a homocysteine.

41. The insulin analogue or complex of any one of claims 1 to 19, wherein the insulin analogue comprises an A-chain polypeptide and a B-chain polypeptide and wherein:
the A-chain and/or the B chain is modified with a glucose-binding moiety or a binding partner moiety, and the cognate binding partner is bound to the glucose-binding moiety or binding partner moiety so as to inhibit binding of the insulin analogue to the insulin receptor.

42. The insulin analogue or complex of claim 41, wherein the cognate binding partner conjugated to a macromolecular carrier optionally through a spacer.

43. The insulin of claim 42, wherein the macromolecular carrier is albumin or an albumin-binding molecule.

44. The insulin analogue or complex of any one of claims 41 to 43, wherein a glucose-binding moiety or a binding partner moiety are attached at or near the N-terminus of the A-chain; and/or a glucose-binding moiety and/or a binding partner moiety are attached at or near C-terminus of the B-chain.

45. The insulin analogue or complex of claim 44, wherein the glucose-binding moiety or the binding partner moiety are attached to a position selected from A1-A10.

46. The insulin analogue or complex of claim 44, wherein the glucose-binding moiety or the binding partner moiety are attached to a position selected from B16-B30.

47. The insulin analogue or complex of any one of claims 41 to 46, wherein a glucose binding moiety or binding partner moiety is linked to a B-chain extension, wherein the extension has an additional B31 amino acid or additional B31 and B32 amino acids.

48. The insulin analogue or complex of claim 47, wherein the amino acid at position B31 is a cysteine or a homocysteine and the amino acid at position B32 is cysteine or a homocysteine.

49. The insulin analogue or complex of any one of claims 1 to 48, where the insulin is a mammalian insulin or a variant thereof.

50. A composition comprising the insulin analogue or complex of any one of claims 1 to 48 and a pharmaceutically acceptable carrier or excipient.

51. The composition of claim 50, wherein the composition is formulated for subcutaneous delivery.

52. A method of lowering the blood sugar of a patient comprising a physiologically effective amount of the insulin analogue or complex according to any one of claims 1 to 48 or a composition according to claim 50 or 51.
